(19) United States
(12) Patent Application Publication
(10) Pub. No.: US 2025/0127876 A1
(43) Pub. Date: Apr. 24, 2025

(54) PORCINE REPRODUCTIVE AND RESPIRATORY SYNDROME VACCINE VIRUS

(71) Applicants: Elanco US Inc., Greenfield, IN (US); Elanco UK AH Limited, Hampshire (GB)

(72) Inventors: Stephen Qitu WU, Fishers, IN (US); Cinta Prieto SUAREZ, Madrid (ES); Geoffrey Gregory LABARQUE, Neuilly sur Seine (FR)

(21) Appl. No.: 19/004,639

(22) Filed: Dec. 30, 2024

Related U.S. Application Data
(63) Continuation of application No. 17/618,625, filed on Dec. 13, 2021, now Pat. No. 12,214,032.

(30) Foreign Application Priority Data
Aug. 29, 2019 (EP) 19382734.2

(51) Int. Cl. A61K 39/12; A61K 39/00; A61P 31/14; C12N 7/00

(52) U.S. Cl. CPC A61K 39/12; A61P 31/14; C12N 7/00; A61K 2039/525; A61K 2039/552

(57) ABSTRACT

The present invention relates to modified, live Porcine Reproductive and Respiratory Syndrome viruses. Viruses were genetically analyzed and selected based on phylogenetic grouping for modification by repeated passage in tissue culture. The modified, live viruses were assessed for the ability to provide protective immunity to heterologous viruses. The modified, live viruses are useful in vaccines, particularly in vaccines which can treat infection of swine by multiple heterologous viruses.

### CROSS-REFERENCE TO RELATED APPLICATIONS

This patent application is a continuation patent application of U.S. patent application Ser. No. 17/618,625, filed 13 Dec. 2021, which is a National Stage entry of International Patent Application No. PCT/EP2019/076174, filed 27 Sep. 2019, which claims priority to European Patent Application No. 19382734.2, filed 29 Aug. 2019.

### REFERENCE TO SEQUENCE LISTING SUBMITTED AS A COMPLIANT XML FILE

Pursuant to the EFS-Web legal framework and 37 C.F.R. § 1.821-825 (see M.P.E.P. § 2442.03 (a)), a Sequence Listing in the form of an xml file (entitled “Sequence_Listing_2920951-236001_ST26.xml” created on 27 Dec. 2024, and 2,587 bytes in size) is submitted concurrently with the instant application, and the entire contents of the Sequence Listing are incorporated herein by reference.

### BACKGROUND

### Field

The present invention relates to modified, live Porcine Reproductive and Respiratory Syndrome viruses. The modified, live viruses are useful in vaccines, particularly in vaccines which provide protection against heterologous viruses.

### Description of Related Art

Porcine Reproductive and Respiratory Syndrome (PRRS), originally called Mystery Swine Disease, was first described in Europe but has now spread worldwide. PRRS causes late-stage abortions, stillbirths, and infertility in breeding age sows, and respiratory disease, decreased growth performance, and even death in nursery and growing/finishing pigs. PRRS causes significant economic losses.

Symptoms of PRRS virus infection in adult porcine animals include, without limitation, reduced appetite, lethargy, and fever. Pregnant sows may prematurely farrow, abort fetuses, or deliver mummified or stillborn piglets, and up to 10% of pregnant sows may die from PRRS virus infection. Infected piglets have a high pre-weaning mortality rate, are often weak, and can have edema around the eyes. PRRS virus infection in weaned nursery or grow/finish pigs can cause, without limitation, a failure to thrive, respiratory distress, labored or rapid breathing, blotchy reddening of the skin, and rough hair coats.

The PRRS virus is an enveloped virus with an approximately 15 kb, linear, positive-stranded RNA genome, and the virus has been classified to the family Arteriviridae. To date at least eleven open reading frames have been identified in the genome. PRRS viruses are divided into two genotypes. The European genotype, Type 1 PRRS viruses (PRRSV-1), are exemplified by the Lelystad strain, while the Type 2 North American PRRS viruses (PRRSV-2) are exemplified by the strain VR-2332.

The two genotypes can have as little as about 60% sequence identity in their genomes, and even within genotypes individual strains can vary up to about 20% in the identity of their genomes. This variability has complicated the development of vaccines to effectively treat and/or prevent PRRS. Modified, live virus (MLV) variants of the PRRS virus can generate immunity against challenge with PRRS viruses, but the vaccine is most effective when the challenge is with a PRRS virus genetically homologous to the MLV. The MLV vaccines have been less effective against challenge with heterologous viruses. Further, MLV have shown some reversion to virulence, such that the vaccine virus causes disease in vaccinated animals. Vaccines containing inactivated (i.e. killed) PRRS viruses have better safety profiles, but efficacy against heterologous challenge has been limited.

Because current PRRS vaccines do not show sufficient safety and efficacy to reduce the economic impact of PRRS virus infection, new and improved vaccines are needed. Preferably, those vaccines would be both safe and efficacious. If the vaccines comprise attenuated MLV, those attenuated MLV should not demonstrate reversion to virulence in order to be considered safe to use in the field. For example, by adapting a PRRS strain to growth in tissue culture cells for at least 60 passages, at least 70 passages, at least 80 passages, or preferably at least 85 passages, the MLV should not demonstrate reversion to virulence. To be efficacious, a vaccine virus strain should be able to elicit protective immunity in a porcine animal against a range of phylogenetically diverse wild type PRRS strains. Preferably, a new PRRS vaccine virus strain would be able to elicit protective immunity in a porcine animal against at least three phylogenetically diverse wild type PRRS strains.

### SUMMARY OF THE INVENTION

The present invention provides for a modified, live Porcine Reproductive and Respiratory Syndrome vaccine virus strain, wherein the consensus complementary DNA sequence of said PRRS strain is at least 90% identical to a sequence selected from the group of SEQ ID NO: 1, SEQ ID NO: 2, SEQ ID NO: 3, and SEQ ID NO: 4. Preferably, the modified, live strain could have a consensus complementary DNA sequence that is at least 95% identical to a sequence selected from the group of SEQ ID NO: 1, SEQ ID NO: 2, SEQ ID NO: 3, and SEQ ID NO: 4. More preferably, the modified, live strain could also have a consensus complementary DNA sequence that is at least 98% identical to a sequence selected from the group of SEQ ID NO: 1, SEQ ID NO: 2, SEQ ID NO: 3, and SEQ ID NO: 4. As a person of ordinary skill will appreciate, due to the high mutation rate of the PRRS virus, a modified, live PRRS strain might comprise a multiplicity of subpopulations, each having a homologous but not identical genome.

The present invention provides for a modified, live Porcine Reproductive and Respiratory Syndrome (PRRS) virus strain, wherein the PRRS virus strain is a DE 14-3073, a ES 13-49, a IT 14-32, or a PL 14-02 strain. The PRRS virus strain should be passaged preferably at least 60 times, or more preferably 70 times, or even more preferably 80 times, in tissue culture cells. Most preferably, the PRRS virus strain should be passaged 85 times in tissue culture cells. Such passaging in tissue culture cells is useful in attenuating the modified, live PRRS virus strain. Attenuated PRRS virus strains may cause subclinical but not clinical disease when those strains are administered to porcine animals. Modified, live PRRS virus strains passaged at least 80 times have a low probability of reverting to wild-type virulence. Most preferably, modified, live PRRS virus strains passaged 85 times have a low probability of reverting to wild-type virulence.

The present invention provides for an immunogenic composition comprising a modified, live PRRS virus strain having a consensus complementary DNA sequence at least 90% identical to a sequence selected from the group consisting of SEQ ID NO: 1, SEQ ID NO: 2, SEQ ID NO: 3, and SEQ ID NO: 4. Preferably, the modified, live strain could have a consensus complementary DNA sequence that is at least 95% identical to a sequence selected from the group consisting of SEQ ID NO: 1, SEQ ID NO: 2, SEQ ID NO: 3, and SEQ ID NO: 4. More preferably, the modified, live strain could also have a consensus complementary DNA sequence that is at least 98% identical to a sequence selected from the group consisting of SEQ ID NO: 1, SEQ ID NO: 2, SEQ ID NO: 3, and SEQ ID NO: 4. As a person of ordinary skill will appreciate, due to the high mutation rate of the PRRS virus, a modified, live PRRS strain might comprise a multiplicity of subpopulations, each having a homologous but not identical genome.

The present invention provides for an immunogenic composition comprising a modified, live PRRS virus strain, wherein said PRRS virus strain is a DE 14-3073 strain, a ES 13-49 strain, a IT 14-32 strain, or a PL 14-02 strain. The DE 14-3073 strain or the ES 13-49 strain or the IT 14-32 strain or the PL 14-02 strain that may be passaged at least 80 times, or preferably even 85 times, in tissue culture cells. Most preferably, the immunogenic composition comprises at least one pharmaceutically-acceptable excipient. The immunogenic composition may also comprise a further antigen from a different virus or from a bacterial strain or from a parasite.

The present invention provides for a vaccine comprising a modified, live PRRS virus strain, wherein the consensus complementary DNA sequence of said PRRS strain is at least 90% identical to a sequence selected from the group consisting of SEQ ID NO: 1, SEQ ID NO: 2, SEQ ID NO: 3, and SEQ ID NO: 4. Preferably, the modified, live strain could have a consensus complementary DNA sequence that is at least 95% identical to a sequence selected from the group consisting of SEQ ID NO: 1, SEQ ID NO: 2, SEQ ID NO: 3, and SEQ ID NO: 4. Most preferably, the modified, live strain could also have a consensus complementary DNA sequence that is at least 98% identical to a sequence selected from the group consisting of SEQ ID NO: 1, SEQ ID NO: 2, SEQ ID NO: 3, and SEQ ID NO: 4. The vaccine may further comprise an adjuvant. The vaccine may further comprise a pharmaceutically-acceptable excipient, stabilizer, solubilizer, or diluent. The vaccine may comprise a further antigen from a different virus or from a bacterial strain or from a parasite.

The present invention provides for a vaccine for use in preventing Porcine Reproductive and Respiratory Syndrome in a porcine animal. As PRRS is caused by a PRRS virus, the present invention provides a vaccine for use in preventing a PRRS virus infection. The present invention also provides for a vaccine for use in a porcine animal for reducing a symptom caused by a PRRS virus infection. The infection may be from a wild-type virulent strain of a PRRS virus. A symptom may be, without limitation, reduced appetite, lethargy, fever, premature farrowing, abortion, stillbirths, edema, a failure to thrive, cough, respiratory distress, labored or rapid breathing, blotchy reddening of the skin, rough hair coats, lung lesions, viral shedding, and mortality. The present invention provides for a vaccine for use in prevention of PRRS in a porcine animal. Preferably, the vaccine comprises a modified, live PRRS strain having a consensus complementary DNA sequence that is at least 90%, at least 95%, or at least 98% identical to a sequence selected from the group consisting of SEQ ID NO: 1, SEQ ID NO: 2, SEQ ID NO: 3, and SEQ ID NO: 4. Most preferably, the vaccine comprises a modified, live PRRS strain which is a DE 14-3073 strain, a ES 13-49 strain, a IT 14-32 strain, or a PL 14-02 strain. The vaccine may further comprise a pharmaceutically-acceptable excipient. The vaccine may further comprise an adjuvant. The vaccine may comprise a further antigen from a different virus or from a bacterial strain or from a parasite.

The present invention provides for a method of preventing a symptom of Porcine Reproductive and Respiratory Syndrome in a porcine animal, comprising administering to said porcine animal an immunogenic composition comprising a modified, live PRRS virus strain. The present invention also provides for a method of preventing Porcine Reproductive and Respiratory Syndrome in a porcine animal, comprising administering to said porcine animal an immunogenic composition comprising a modified, live PRRS virus strain. Preferably, the modified, live PRRS virus strain for use in the method would have a consensus complementary DNA sequence at least 90% identical to a sequence selected from the group consisting of SEQ ID NO: 1, SEQ ID NO: 2, SEQ ID NO: 3, and SEQ ID NO: 4. More preferably, the modified, live PRRS virus strain for use in the method would have a consensus complementary DNA sequence that is at least 95% identical to a sequence selected from the group consisting of SEQ ID NO: 1, SEQ ID NO: 2, SEQ ID NO: 3, and SEQ ID NO: 4. Most preferably, the modified, live PRRS virus strain for use in the method would have a consensus complementary DNA sequence that is at least 98% identical to a sequence selected from the group consisting of SEQ ID NO: 1, SEQ ID NO: 2, SEQ ID NO: 3, and SEQ ID NO: 4. The immunogenic composition may further comprise a pharmaceutically-acceptable excipient. The immunogenic composition may comprise a further antigen from a different virus or from a bacterium or from a parasite.

The present invention provides for a method of preventing Porcine Reproductive and Respiratory Syndrome in a porcine animal, comprising administering to said porcine animal an immunogenic composition comprising a modified, live Porcine Reproductive and Respiratory Syndrome (PRRS) virus strain, wherein the said PRRS virus strain is a DE 14-3073 strain, a ES 13-49 strain, a IT 14-32 strain, or a PL 14-02 strain for use in the method may be passaged at least 80 times, or preferably even 85 times, in tissue culture cells. The immunogenic composition may further comprise a pharmaceutically-acceptable excipient. The immunogenic composition may comprise a further antigen from a different virus or from a bacterial strain or from a parasite.

The present invention provides for a method of preventing a symptom caused by a PRRS virus infection in a porcine animal, comprising administering to said porcine animal an immunogenic composition comprising a modified, live Porcine Reproductive and Respiratory Syndrome (PRRS) virus strain, wherein the consensus complementary DNA sequence of said PRRS strain is preferably at least 90% identical to a sequence selected from the group consisting of SEQ ID NO: 1, SEQ ID NO: 2, SEQ ID NO: 3, and SEQ ID NO: 4. More preferably, the modified, live strain for use in the method could also have a consensus complementary DNA sequence that is at least 95% identical to a sequence selected from the group consisting of SEQ ID NO: 1, SEQ ID NO: 2, SEQ ID NO: 3, and SEQ ID NO: 4. Most preferably, the modified, live strain for use in the method could also have a consensus complementary DNA sequence that is at least 98% identical to a sequence selected from the group consisting of SEQ ID NO: 1, SEQ ID NO: 2, SEQ ID NO: 3, and SEQ ID NO: 4. The immunogenic composition may further comprise a pharmaceutically-acceptable excipient. The immunogenic composition may also comprise a further antigen from a different virus or from a bacteria strain or from a parasite.

The present invention provides for a method of reducing a symptom caused by a PRRS virus infection in a porcine animal, comprising administering to said porcine animal an immunogenic composition comprising a modified, live Porcine Reproductive and Respiratory Syndrome (PRRS) virus strain, wherein the said PRRS virus strain is a DE 14-3073 strain, a ES 13-49 strain, a IT 14-32 strain, or a PL 14-02 strain for use in the method may be passaged at least 80 times, or preferably even 85 times, in tissue culture cells. The immunogenic composition may further comprise a pharmaceutically-acceptable excipient. The immunogenic composition may also comprise a further antigen from a different virus or from a bacteria strain or from a parasite. The PRRS virus infection may be an infection by a virulent PRRS virus heterologous to the modified, live PRRS virus strain in the immunogenic composition. Two PRRS virus strains are considered to be heterologous if a genomic consensus sequence of each virus strain maps to a different phylogenetic group. Two PRRS virus strains are considered to be heterologous if a complementary DNA consensus sequence of each virus strain maps to a different phylogenetic group.

The present invention provides for the use of a modified, live PRRS virus strain in the manufacture of a medicament for preventing or reducing a symptom of PRRS, wherein the modified, live PRRS virus comprises a consensus complementary DNA sequence at least 90% identical to a sequence selected from the group consisting of SEQ ID NO: 1, SEQ ID NO: 2, SEQ ID NO: 3, and SEQ ID NO: 4. Preferably, the modified, live strain could also have a consensus complementary DNA sequence that is at least 95% identical to a sequence selected from the group of SEQ ID NO: 1, SEQ ID NO: 2, SEQ ID NO: 3, and SEQ ID NO: 4. More preferably, the modified, live strain could also have a consensus complementary DNA sequence that is at least 98% identical to a sequence selected from the group consisting of SEQ ID NO: 1, SEQ ID NO: 2, SEQ ID NO: 3, and SEQ ID NO: 4.

The present invention provides for the use of a modified, live PRRS virus strain comprising a DE 14-3073 strain, a ES 13-49 strain, a IT 14-32 strain, or a PL 14-02 strain in the manufacture of a medicament for preventing or reducing a symptom of PRRS. The modified, live PRRS virus strain should be passaged at least 80 times, or preferably even 85 times, in tissue culture cells. Such passaging in tissue culture cells is useful in properly attenuating the modified, live PRRS virus strain. Attenuated PRRS virus strains may cause subclinical but not clinical disease when those strains are administered to porcine animals. Modified, live PRRS virus strains passaged at least 80 times have a low probability of reverting to wild-type virulence.

The present invention provides for the use of an immunogenic composition comprising a modified, live PRRS virus strain in the manufacture of a medicament for preventing a PRRS virus infection, wherein the modified, live PRRS virus strain comprises a consensus complementary DNA sequence at least 90% identical to a sequence selected from the group consisting of SEQ ID NO: 1, SEQ ID NO: 2, SEQ ID NO: 3, and SEQ ID NO: 4. The modified, live strain for such use could also have a consensus complementary DNA sequence that is at least 95% identical to a sequence selected from the group consisting of SEQ ID NO: 1, SEQ ID NO: 2, SEQ ID NO: 3, and SEQ ID NO: 4. The modified, live strain for such use could also have a consensus complementary DNA sequence that is at least 98% identical to a sequence selected from the group consisting of SEQ ID NO: 1, SEQ ID NO: 2, SEQ ID NO: 3, and SEQ ID NO: 4.

The present invention provides for the use of an immunogenic composition comprising a modified, live PRRS virus strain comprising a DE 14-3073 strain, a ES 13-49 strain, a IT 14-32 strain, or a PL 14-02 strain in the manufacture of a medicament for preventing a PRRS virus infection. The PRRS virus strain should be passaged at least 80 times, or preferably even 85 times, in tissue culture cells. Such passaging in tissue culture cells is useful in properly attenuating the modified, live PRRS virus strain. Attenuated PRRS virus strains may cause subclinical but not clinical disease when those strains are administered to porcine animals. Modified, live PRRS virus strains passaged at least 80 times have a low probability of reverting to wild-type virulence. Modified, live PRRS virus strains passaged 85 times have a low probability of reverting to wild-type virulence.

The present invention provides for the use of an immunogenic composition comprising a modified, live PRRS virus strain in the manufacture of a medicament for protecting a porcine animal from a PRRS virus infection, wherein the modified, live PRRS virus strain comprises a consensus complementary DNA sequence at least 90% identical to a sequence selected from the group consisting of SEQ ID NO: 1, SEQ ID NO: 2, SEQ ID NO: 3, and SEQ ID NO: 4. The modified, live strain for such use could also have a consensus complementary DNA sequence that is at least 95% identical to a sequence selected from the group consisting of SEQ ID NO: 1, SEQ ID NO: 2, SEQ ID NO: 3, and SEQ ID NO: 4. The modified, live strain for such use could also have a consensus complementary DNA sequence that is at least 98% identical to a sequence selected from the group consisting of SEQ ID NO: 1, SEQ ID NO: 2, SEQ ID NO: 3, and SEQ ID NO: 4.

The present invention provides for the use of an immunogenic composition comprising a modified, live PRRS virus strain comprising a DE 14-3073 strain, a ES 13-49 strain, a IT 14-32 strain, or a PL 14-02 strain in the manufacture of a medicament for protecting a porcine animal from a PRRS virus infection. The PRRS virus strain should be passaged at least 80 times, or preferably even 85 times, in tissue culture cells. Such passaging in tissue culture cells is useful in properly attenuating the modified, live PRRS virus strain. Attenuated PRRS virus strains may cause subclinical but not clinical disease when those strains are administered to porcine animals. Modified, live PRRS virus strains passaged at least 80 times have a low probability of reverting to wild-type virulence. Modified, live PRRS virus strains passaged 85 times have a low probability of reverting to wild-type virulence.

### BRIEF DESCRIPTION OF THE DRAWINGS

FIG. 1. Phylogenetic analysis of PRRSV-1 a DE 14-3073 strain, a ES 13-49 strain, a IT 14-32 strain, or a PL 14-02 strain and commercially available vaccines and selected isolates based on ORF5 nucleotide sequences constructed by the neighbor joining method.

### DETAILED DESCRIPTION OF THE INVENTION

As used in the following discussion, the terms “a” or “an” should be understood to encompass one or more, unless otherwise specified.

As used herein, the term “virus” could mean either the species of virus, or, interchangeably, an individual infectious unit which may contain nucleic acids and proteins. An individual infectious unit is also called a “viral particle” or a “virion”, the latter terms being synonymous.

As used herein, a “strain” or “isolate” a virus means a collection of genetically homologous virions. Two viruses would be considered “homologous” if those viruses map to the same phylogenetic clade. Two viruses would be considered “heterologous” if those viruses map to different phylogenetic clades. As the PRRS virus has a high mutation rate, it will be appreciated that a single PRRS strain comprises individual virions with related but variable genetic sequences. Thus, subpopulations of strains exist within each PRRS strain, and the genetic sequence of a PRRS strain is a consensus sequence such that the genetic sequence of an individual member of the PRRS strain may not be identical to the consensus sequence for that strain. A “consensus” sequence is a nucleic acid sequence in which each nucleic acid residue at a given position is present in >51% of the polynucleotides in a PRRS virus strain or isolate.

“Percent identity” can be determined by calculating the number of identical nucleotides or amino acids at the same positions in a nucleic acid or protein. Calculation of percent identity includes determination of the optimal alignment between two or more sequences. Alignment can take into account insertions and deletions (i.e. “gaps”) in each of the sequences to be tested, such as, without limitation, in the non-coding regions of nucleic acids and truncations or extensions of polypeptide sequences. Computer programs and algorithms such as the Basic Local Alignment Search Tool (BLAST) may be used to determine the percent identity. BLAST is one of the many resources provided by the U.S. National Center for Biotechnology Information. Because the genetic code is degenerate, and more than one codon can encode a given amino acid, coding regions of nucleic acids are considered identical if the nucleic acids encode identical polypeptides. Thus, percent identity could also be calculated based on the polypeptide encoded by the nucleic acid. Percent identity could be calculated based on full length consensus genomic sequences or on a fraction of the genomic sequence, such as for example without limitation on individual open reading frames (ORFs).

As used herein, the term “modified, live virus” applies to any individual viral particle (i.e. a “virion”) or to a multiplicity of viral particles whose genetic sequence has been altered from the genetic sequence of a naturally-occurring wild type virus yet still elicits protective immunity against wild type virus. Alterations include, without limitation, genetic mutations such as insertions and deletions of nucleotides and transitions and transversions which change one nucleotide for another nucleotide. Alterations can be accomplished by adapting a wild-type virus to replication in a tissue culture system, and continuing to passage a virus in a tissue culture system, whereby the virus accumulates genetic mutations. Alterations can also be accomplished using molecular techniques. Attenuated viruses form a subset of modified, live viruses.

As used herein, the term “attenuated” or “attenuation” means the ability of virus to cause or exacerbate clinical disease has been reduced or eliminated. An attenuated virus can still infect a host cell, either in vitro or in vivo, and that infection may result in subclinical effects in the host organism, but that infection does not result in one or more clinical disease symptoms.

In contrast, as used herein, “inactivated” viruses mean viruses which can no longer replicate in a host cell. Inactivated viruses are considered to be killed or dead viruses. Inactivation can be accomplished by a variety of methods, including but not limited to chemical alteration of viral proteins, to chemical or physical alterations in the structure of a virion, or to chemical or physical alterations in viral nucleic acids.

An “antigen” is any molecule capable of being specifically detected by the immune system of an organism. Typically a viral antigen is a viral protein encoded by the viral genome or derived from products of the viral genome. The presence of viral antigens can be specifically detected by the surface antigen receptors of both host T lymphocytes and host B lymphocytes and by antibody molecules synthesized by host cells.

“Immunogenicity” refers to the ability of an antigen to elicit an immune response, said immune response comprising both antigen-specific responses and non-antigen-specific responses or innate immune responses. “Protective immunity” is an immune response which can reduce or prevent clinical symptoms when an immunized animal is challenged or exposed to a pathogenic virus strain. As one skilled in the art would appreciate, protective immunity may decline with time or increased age of the immunized animal. Protective immunity as used herein should be effective for at least four months, but preferably at least six months, from the latest date of immunization. Protective immunity may be elicited with a single dose of a vaccine. A second or further dose may be used to increase or prolong the protective immune response. For example, increasing the protective immune response in a breeding sow may result in an increased level of maternally derived antibody in piglets.

In contrast to an antigen, an “adjuvant” is a non-specific stimulator of an immune response. An adjuvant could stimulate the innate immune response by binding and activating a pattern recognition receptor (PRR). Such stimulators of PRRs could be, for example, viral or bacterial nucleic acids, lipids from bacteria or parasites, or bacterial proteins or toxins, or any artificially-constructed mimic of such molecules. Adjuvants also include, without limitation: inorganic compounds that aggregate antigens to facilitate recognition by B lymphocytes or uptake by phagocytes, such as alum, aluminum hydroxide, aluminum phosphate, calcium phosphate hydroxide or ammonium sulfate; oils; and detergents. Adjuvants could also be host mediators of immune signaling, such as, without limitation, cytokines, lymphokines, chemokines, interferons, anaphylatoxins, growth factors, differentiation factors, and adhesion molecules.

As used herein, an “immunogenic composition” is a composition that elicits an immune response when administered to an animal. An immunogenic composition comprises at least one antigen and at least one pharmaceutically-acceptable excipient. The antigen can be a whole virus, bacterium, or other pathogen, either live or inactivated. The antigen can also be isolated, purified, or partially purified antigenic molecule from a virus, bacterium, or other pathogen. The antigen can be a polypeptide, a polysaccharide, a nucleic acid, or a lipid.

As used herein, a “vaccine” is an immunogenic composition which confers protection from, resistance to, prevention of, or reduction for a disease symptom when administered to an animal, wherein said symptom is caused by a pathogenic organism, for example a virus. A PRRS vaccine may include, without limitation, viral antigens or intact virions, either live or inactivated, in composition with at least one pharmaceutically-acceptable excipient.

As used herein, the terms “treating”, “to treat”, or “treatment”, include without limitation restraining, slowing, stopping, reducing, ameliorating, or reversing the progression or severity of an existing symptom, disorder, condition, or disease. A treatment may be applied or administered therapeutically.

As used herein, the terms “preventing”, “to prevent”, or “prevention”, include without limitation decreasing, reducing, or ameliorating the risk of a symptom, disorder, condition, or disease, and protecting an animal from a symptom, disorder, condition, or disease. A prevention may be applied or administered prophylactically.

As used herein, “administering to an animal” includes but is not limited to cutaneous, subcutaneous, intramuscular, mucosal, submucosal, transdermal, oral or intranasal administration. Administration could include injection or topical administration.

The term “pharmaceutically-acceptable excipient” refers to refers to those typically used in preparing veterinary and pharmaceutical compositions and should be pure and non-toxic in the amounts used. In certain embodiments, the pharmaceutical composition may contain excipients for modifying, maintaining or preserving, for example, the pH, osmolarity, viscosity, clarity, isotonicity, sterility, stability, adsorption or penetration of the composition. Some examples of acceptable excipients are found in, for example, Remington's Pharmaceutical Sciences and the Handbook of Pharmaceutical Excipients, 18^{th} Edition, (A. R. Genrmo, ed.), 1990, Mack Publishing Company and later editions and Remington: The Science and Practice of Pharmacy, Lloyd V. Allen, ed., Pharmaceutical Press, London, UK, 22^{nd} edition, 2012. and include diluents, vehicles, carriers, stabilizing agents, preservatives, solvents, suspending agents, emulsifiers, antimicrobials, antioxidants, buffers, chelating agents, complexing agents, carbohydrates, proteins, diluting agents, and/or pharmaceutical adjuvants. In certain embodiments, the primary vehicle or carrier in a pharmaceutical composition may be either aqueous or non-aqueous in nature. For example, a suitable vehicle or carrier may be water for injection, physiological saline solution with other materials common in compositions for parenteral administration.

As used herein, the term “porcine animals” refers to pigs, any of the animals in the genus Sus within the even-toed ungulate family Suidae.

The following experimental examples are illustrative of modified, live PRRS viruses. The following experimental examples are also illustrative of immunogenic compositions comprising modified, live PRRS viruses. The following experimental examples are also illustrative of using modified, live PRRS viruses to prevent or reduce porcine animals for symptoms of PRRS. It will be appreciated that other embodiments and uses will be apparent to those skilled in the art and that the invention is not limited to these specific illustrative examples or preferred embodiments.

### Example 1

The objective of this study was to identify potential strains for development of vaccines. A total of 36 PRRS Type 1 field isolates have been evaluated for their potential as vaccines. Below is the preparation of pre-master seed viruses (Pre-MSVs) of four type 1 (European) PRRS virus (PRRSV-1) strains: DE 14-3073, ES 13-49, IT 14-32, and PL 14-02.

Initial isolation for PRRSV-1 strains was done using serum and lung tissues originating from pig herds diagnosed by PRRSV-positive test results during 2013 to 2014 in Europe. The pig herds were not vaccinated against PRRSV but experienced clinical signs characteristic for PRRSV infections, including reproductive failure in pregnant gilts and sows (i.e. late-term abortions, early farrowings, birth of weak and stillborn piglets and/or increased pre-weaning mortality) and/or growth retardation and respiratory disease problems in pigs of young ages.

Virus isolation was carried out in primary cultures of porcine alveolar macrophages (PAMs). To obtain PAM cultures 3-week-old piglets were used as donors. Briefly, lungs were obtained in aseptic conditions after the piglets were humanely euthanized and then flushed with phosphate buffer saline to retrieve PAM cells. The cell suspension obtained was centrifuged at 800×g for 15 minutes at 4° C. and the supernatant discarded. Pelleted cells were resuspended in Dulbecco's modified Eagle's medium (DMEM) and washed twice using DMEM as diluent after the above-mentioned centrifugation conditions.

The cells were counted and seeded in different supports (i.e. cellular culture flasks or plates of different sizes) at the final concentration of 3×10⁶ cells/mL in DMEM supplemented with 10% fetal bovine serum (FBS) and antibiotic-antimycotic solution (100 units/mL of penicillin, 100 μg/mL of streptomycin and 0.25 μg/mL of amphotericin B). The cells were cultured at 37° C. in an atmosphere with 5% CO₂.

Clinical samples were processed following different procedures depending on their nature. Thus, serum samples were filtered through a 0.22 μm sterile syringe filter and kept at −80° C. until used for virus isolation. On the other hand, tissue samples, including tonsils and lungs, were homogenized 1:10 using DMEM as diluent. The homogenates were clarified by centrifugation at 2500×g for 15 minutes and the supernatants were filtered through a 0.22 μm sterile syringe filter and kept at −80° C. until used for virus isolation.

To infect PAM cultures the media was removed and the clinical samples (i.e. processed serum samples or processed tissue samples) were added in variable amounts depending on the support used. After 1.5 hours at 37° C. for adsorption, the cultures were washed and fresh DMEM supplemented as for cell maintenance was added. Cultures were observed daily for cytopathic effect (CPE). When CPE was observed, the cultures were harvested. After 3 cycles of freezing and thawing, cell debris was removed by centrifugation at 2500×g at 4° C. for 15 min and the supernatant was frozen and stored at −80° C. The presence of porcine reproductive and respiratory syndrome virus (PRRSV) in the culture was confirmed by reverse transcription and polymerase chain reaction (RT-PCR).

When no CPE was observed the culture was considered negative and the original clinical samples (i.e. serum samples or tissue samples) were used in a bioassay and inoculated to 3-week-old piglets housed in isolation. For this purpose, clinical samples were filtered and injected, by the intramuscular route, to the pigs. After one week, blood samples were taken from the exposed pigs to confirm viremia by RT-PCR. Pigs confirmed to be viremic were euthanized and blood, tonsil and lung samples were collected at necropsy and used as inocula for a second attempt of virus isolation in PAM cultures following the method previously described.

The growth of the PRRSV isolates in the MARC-145 cell line was attempted only for those isolates which grew well in PAMs and for which a seed stock could be produced. For this purpose, MARC-145 cells seeded in 25 cm² cell culture flasks at a concentration of 5×10⁵ cells/flask and maintained at 37° C. in at atmosphere with 5% CO₂ in DMEM supplemented with 10% FBS and antibiotic-antimycotic solution (100 units/mL of penicillin, 100 μg/mL of streptomycin and 0.25 μg/mL of amphotericin B). Pre-confluent cultures were infected with the supernatants of positive PAM cultures following the same protocol described for the infection of PAM cultures. After adsorption, cells were washed and cultured in DMEM supplemented with 5% FBS and the previously mentioned antibiotic-antimycotic solution.

After the initial isolation, the viruses were amplified and primary virus stock (with a volume of at least 100 mL) were produced for each isolate in PAM cultures. For this purpose, PAM cells were cultured for 24 h at 37° C. in an atmosphere with 5% CO₂ in 75 cm² bottles at a concentration of 3×10⁶ cells/mL in DMEM supplemented with 10% FBS and the antibiotic-antimycotic solution previously described. Then the medium was discarded, the cells were washed with fresh DMEM and the viral inocula were added. After 1.5 hours at 37° C. for adsorption, the cultures were washed and fresh DMEM supplemented as for cell maintenance was added. Cultures were observed daily for CPE. When most cells in a culture were killed, the cultures were harvested. After 3 cycles of freezing and thawing, cell debris was removed by centrifugation at 2500×g at 4° C. for 15 min and the supernatant containing virus was frozen and stored at −80° C. Virus titrations were calculated according to the method of the Reed and Muench (1938) and expressed as log TCID₅₀/mL. Viral stocks were kept at −80° C. and used in the different objectives of the study.

The same procedure were used with MARC-145 cell cultures. MARC-145 cell cultures were inoculated as previously described for initial isolation and maintained at 37° C. in an atmosphere of 5% CO₂. When CPE was evident (i.e. affecting approximately 70-80% of the monolayer) or after 5 days of culture if CPE is not observed, cultures are subjected to three cycles of freezing and thawing, clarified by centrifugation at 2500×g for 15 minutes and the supernatants used to inoculate fresh MARC-145 cell cultures. The remaining supernatant of each passage was stored at −80° C. A total of 60 passages in the MARC-145 cell line were undertaken for each PRRSV isolate.

On passages 30 and 50 viral stocks were cloned by plaque purification, following a standard methodology. Briefly, 6-well plates previously seeded with MARC-145 were inoculated when the cultures were pre-confluent with serial dilutions of each viral stock, from 10¹ to 10⁶. After 1.5 hours of adsorption, the inoculum was removed from each well and the cells were covered with fresh DMEM media supplemented with 5% FBS, the abovementioned antibiotic-antimycotic solution and 1% low melting agarose. After 2-4 days of culture, depending on the isolate, individual plaques were selected under a phase contrast microscope and picked up. At least five plaques were selected for each virus in each purification round based on their complete isolation in the monolayer to guarantee the clonal nature of the selected viruses. Selected plaques were used as inocula for the next purification round. The procedure was repeated three times to assure that the obtained viral progeny derived from one single virus.

### Example 2

The objective of this study was to further characterize the PRRS-1 isolates. The portion of ORF1 coding for nsp2 and ORFs 2 to 7 were amplified by RT-PCR using a set of primers previously designed. For this purpose, total RNA was obtained from all viral stocks using QIAMP® Viral RNA Mini kit (Qiagen, USA) following the manufacturer's instructions. For reverse transcription and polymerase chain reaction (RT-PCR), 15 μL of total RNA were used as template. The reaction was performed using a commercial one step RT-PCR kit (SuperScript III OneStep RT-PCR PLATINUM TAQHIFI®, Invitrogen, USA), following the manufacturer's instructions. RT-PCR products were purified using a commercial kit (QIAQUICK® Purification Gel Kit, Qiagen, USA) following the manufacturer's instructions. Individual sequences of both strands of DNA of each PCR product were determined using the same pair of primers used for RT-PCR, amplifying the samples by asymmetric PCR with fluorescent terminators and analyzing the products by electrophoresis on an ABI prism 310 Genetic Analyzer (Applied Biosystems, USA). At least two different RT-PCR products were sequenced to verify that no errors had occurred during DNA amplification and that the sequence obtained was correct. Sequences were manually corrected, purged of errors and aligned using Clustal Omega software. The sequences obtained were compared to the genotype 1 PRRSV prototype Lelystad virus and the vaccine strains which constitute the basis of the vaccines PORCILIS® PRRS (MSD Animal Health) (DV strain), UNISTRAIN® PRRS (Laboratorios Hipra) (VP-046 BIS strain) and INGELVAC PRRSFLEX® EU (Boehringer Ingelheim) (strain 94881). In addition they have been compared to the genotype 2 prototype strain VR-2332, which is the strain of INGELVAC PRRS® MLV vaccine (Boehringer Ingelheim), also commercially available in Europe.

The nucleotide similarity among the PRRSV isolates obtained was calculated as well as the similarity between each field isolate and the vaccine strains available at the time of isolation with the objective to confirm that the isolates were not related to one another and that they were not derivatives of commercially available vaccines at the time of clinical sample collection. Additionally, a phylogenetic tree was constructed using the neighbor joining method and including VR-2332, the prototype of the American genotype, as an outgroup to determine the subtype to which the Eutopean isolates belonged. To assess the statistical reliability of the dendrograms, bootstrapping values were calculated (random number seed: 123; 1,000 replicates). All phylogenetic analyses were performed with MEGA 5.0 software.

The PRRSV-1 was further attenuated by passing 10 times (to P70) in MARC-145 cells in the growth medium OPTI-MEM® | (Cat No. 31985, Life Technologies,) supplemented with 2% fetal bovine serum (FBS; Cat No. 12003C and 12007C from Sigma and Cat No. 04-4000DJ from Gibco) and 50 μg gentamicin/mL (Cat No. 15750, Life Technologies) and by additionally passing 15 more times (to P85) in the same growth medium supplemented with 2% FBS without gentamicin. The identity of the 85th passage (P85) PRRSV-1 was confirmed by indirect immunofluorescence assay (IFA) using PRRSV-specific monoclonal antibody and the P85 PRRSV-1 was considered as the Pre-Master Seed Viruses (pre-MSV).

The following procedure was used to determine the titer of PRRSV-1. MARC-145 cells were seeded into 96-well plates at a density of 0.75 to 1.5×10⁴ cells in 100 μL of growth medium (OPTI-MEM® | media supplemented with 5% FBS and 50 μg/mL gentamycin). Cells were incubated in 37±2° C. and 5±1% CO₂ incubator for 48-72 hours until cells were over 95% confluent. On the day of titration, all media was removed from the 96-well plate and replaced with 100 μL of fresh growth media.

Ten-fold serial dilutions of the PRRSV-1 were prepared with diluent (OPTI-MEM® | media, 50 μg/mL gentamycin) and transferred to corresponding wells on the plates prepared as above along with a negative control consisting of diluent alone and a positive control with a known titer. Titration plates were incubated in 37±2° C. with 5±1% CO₂ incubator for 4 days. At the end of the incubation period, each plate was observed for the presence of virus-induced cytopathic effect (CPE) in each sample well using an inverted microscope. The 50% tissue culture infectious dose (TCID₅₀) was calculated using the Reed-Muench method and titer was recorded as log₁₀ TCID₅₀/mL. The titer range of P85 PRRSV-1 was 8.1-8.5 log₁₀ TCID₅₀/mL.

### Example 3

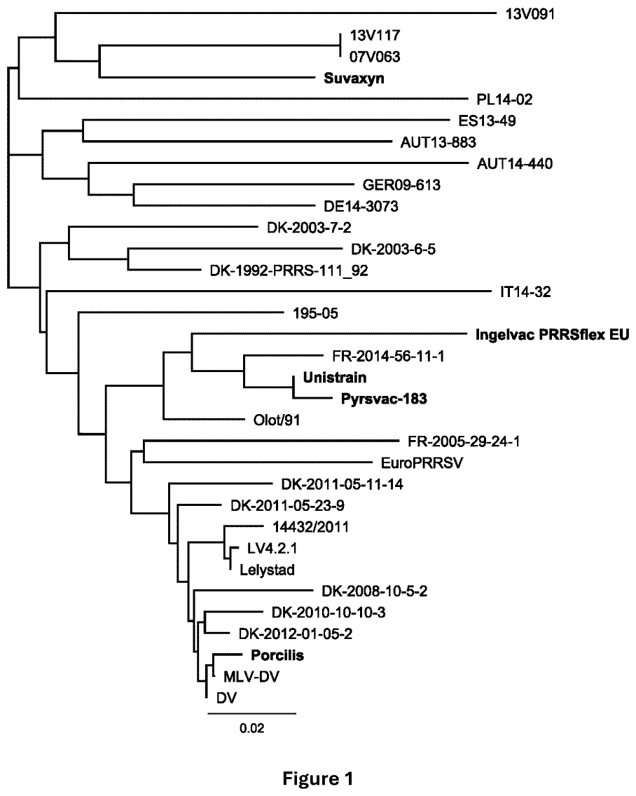

The objective of this study was to determine the sequences of pre-MSV. To determine the genomic sequence of each virus isolate, viruses grown in MARC-145 cells were concentrated and purified by ultracentrifugation over a sucrose cushion and RNA was extracted using a MINELUTE Virus Spin Kit (Qiagen) TRIZOL LS (Invitrogen). The full genome sequence was determined by multiple runs of next-Gen sequencing (NGS) using ILLUMINA® MISEQ® platform and/or NEXTSEQ500 system at Bioreliance (Rockville, MD) and ACGT (Wheeling, IL). Variable and uncertain sequences and gaps were fixed and confirmed by Sanger dideoxy sequencing and consensus full genome sequences were generated. The nucleotide sequences were aligned and compared to selected known PRRSV-1 and commercial vaccine viruses by neighbor-joining tree nucleotide alignment tool using the software Geneious 10.1.3. (FIG. 1).

The PRRSV-1 MLV strains denoted as Pre-MSVs are deposited under conditions that will assure that access to the cultures during the pendency of this patent application to one determined by the Commissioner of Patents and Trademarks to be entitled thereto under 37 C.F.R. § 1.14 and 35 U.S.C. § 122. The deposit will be available as required by foreign patent laws in countries wherein counterparts of the subject application, or its progeny, are filed. However, it should be understood that the availability of a deposit does not constitute a license to practice the subject invention in derogation of patent rights granted by governmental action. The subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the deposited culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it. A deposit of PRRSV-1 Pre-MSVs was entered into the permanent collection of the Patent Depository of the American Type Culture Laboratory, located at 10801 University Blvd., Manassas, Va., 20110-2209, USA, on Mar. 7, 2019 under the terms of the Budapest Treaty, whereupon the strains were assigned accession numbers PTA-125490 (DE 14-3073); PTA-125489 (ES 13-49); PTA-125488 (IT 14-32) PTA-125487 (PL 14-02); by the repository.

The safety and efficacy of immunogenic compositions and vaccines of modified, live Porcine Reproductive and Respiratory Syndrome viruses can be determined by methods well known in the art, including the dose response, onset of immunity, duration of immunity, and the shedding and transmission of a porcine reproductive and respiratory syndrome virus. The lack of reversion-to-virulence of any Pre-MSV can also be readily determined.

The cDNA consensus sequences for four PRRS virus isolates at passage 85 (P85) were deposited in GenBank genetic sequence database, an annotated collection of all publicly available nucleic acid sequences. The GenBank database is maintained by the National Center for Biotechnology Information (NCBI), part of the United States National Institutes of Health (NIH). GenBank is part of the International Nucleotide Sequence Database Collaboration.

The cDNA consensus sequence of PRRS strain DE14-3073 at P85 has been assigned GenBank Accession number MK024324 (SEQ. ID. NO: 1). The cDNA consensus sequence designated SEQ. ID. NO: 1 is:

| | |
|---|---|
| atgatgtgta gggtattttc cctacgtgcg caacactttt tgtgtttgtg tgcctcggag | 60 |
| gcgtgggtat agccccgccc cacctcttgg ccctgttcta gcccaacagg tatccttctc | 120 |
| tctcggggcg agtgcgccgc ctgctgcttc cttgcagcgg gaaggacctc ccgagtactt | 180 |
| ccggagagcg cctgctttac gggatctcca ccctttaacc atgtctggga cgttctcccg | 240 |
| gtgcatgtgc accccggcgg ctcgggtgtt ttggaacgcc ggccaagtct tttgcacacg | 300 |
| gtgtctcagt gcacggtctc ttctctctcc agagcttcag gacctcagtt ttggtgcact | 360 |
| cggcttgttt tacaagccta aagataagct tcattggaaa gttcctgttg gtatacctca | 420 |
| ggtagagtgc actccatccg ggtgctgttg gctttcagct attttccctc tggcacgtat | 480 |
| gacctccggc aaccacaact ttctccagag acttgtgaag gttgctgacg ttttgtaccg | 540 |
| tgatggttgc ttggcgcccc gacatcttcg tgaacttcaa gtttacgagc gcggctgcag | 600 |
| ttggtacccg attacgggac ccgtgcccgg gatgggtctg tttgcgaatt ccatgcacgt | 660 |
| atccgaccag cctttccctg gtgccaccca tgtgctgact aactcgcctc tgcctcaaca | 720 |
| ggcttgtcgg cagccttttt gcccatttga ggaggctcat tctggtttgt tcaagtggaa | 780 |
| taaattcgtg atttttatag acccccccct taacggtaga cgccgcatga tgtgggcacc | 840 |
| tgaatccgac gattcagcca acttggaggt gttgccgcct gaattagaac gtcaagttga | 900 |
| gattctcact cggagtttcc ctgctcacca ccctgtcaac ctcagcgact gggagctttc | 960 |
| cgactcccct gaacacggtt tttccttcag cacttatcat tcttctggtt acgttgccaa | 1020 |
| aaaccctgac gtgtttgata gcaagtgctg gctttcctgt tttctgagct tgtcgcctga | 1080 |
| ggtgtggcat cgtgaggagc tcttggctag cgcatttggt tatcaaacca agtggggcgt | 1140 |
| gcatggcaaa tacctccaac gcaggcttca aattaatggc atccgtgctg tggttgatcc | 1200 |
| agacggcccc attcacgttg aagcgctgtc ttgcccccag tcttggatca ggcatttgac | 1260 |
| tctggacgat gaagtaaccc cagggttcgt tcgcctgacg tctcttcgca tcgtaccgaa | 1320 |
| cacagaacct accacctttc gcgtctttcg ttttggggcg cataagtggt atggtgccgc | 1380 |
| cggtaaacga gctcgcgcaa agcgtgctgc caagagcgag aaggactcgg ttactgcatc | 1440 |
| caaggctgtc caaccaactc ttgcctgtaa aaacaccacc tattccccac caacggacgg | 1500 |
| gtcttgtggt tggcatgttc tcgccgccat aatgaaccgg atgttaaatg gtgacttcac | 1560 |
| gtccccttta accccgtaca acagaccaga agatgattgg gcatcggatt atgatcttgc | 1620 |
| tcaggcgatt caatgtctac aattacctgc taccatagtc cggaatcgcg cctgtcctaa | 1680 |
| tgctaaatac ctcataaaac tcaacggagt tcactgggag gtagaggcaa ggtctgggtt | 1740 |
| tgcccctcgc tcccttcccc gcgagtgtgt ggtcggtgtt tgctccgaag gctgtgtcgc | 1800 |
| aacaccctac ccagagaatg ggctgcctaa gcgggcactt gaggccttag cgtctgctta | 1860 |
| cagattgcct tccgattgcg tcagttctgg cattgctgac tttcttgctg acccccctcg | 1920 |
| ggagttttgg actcttgaca aaatgctgac ttccccatca ccggagcaat ccggcttttc | 1980 |
| tagtctgtac agattgctat tagaggttgt cccgcaaaaa tgcggtgcca cggagggggc | 2040 |
| tttcatctat gccgttgaaa ggatgttgaa ggattgtcca agttccaagc aggccatggc | 2100 |
| tctcctagca aaagtcaaag tcccatcctc aaaggcctcg tctgtgacct tggatgagtg | 2160 |
| ttttcctacg gacgttccgg ccgacttcaa gccagcgtct catgagaagc ctcaaagttc | 2220 |
| cggtactgtt gttgtcctgt gttcaccggg agcagaagag tcagaaaaag tgaccctaga | 2280 |
| agaagttcgg gagggtggct ataaaaccat ctgccctgca ccccttactg agggtcctaa | 2340 |
| tgatgaacag gcacaagtag ctgtcggcga gcagctgagg ctcagcggtt gtggtttggc | 2400 |
| agccgggaac gctccagccc cggctggtcc aattgacaca gtaagcagag atcttcccct | 2460 |
| tccggacttc atgaaagaaa acatgtccaa taattgggag gatggaccat tggatttgtc | 2520 |
| ccaatcggca tcagctgtca tgacaacccc tgtaggagag cacacatcca aaaatccagg | 2580 |
| ttctggtatc ggtgacttcc ctgttactgt tcgaggcttt acctcaacgg ggctcgtact | 2640 |
| tcgtcacgtt gagcactgcg gtacggagtt gggcgacgac agtccgcctt tggatttgtc | 2700 |
| tgattcgcag acctcgaacc ggcctctgga tctatcccta gctgcttggc cagtgaagac | 2760 |
| caccgcatct gaccctggct gggttcatgg tagacgcgaa cctgtctttg taaagcctcg | 2820 |
| gaatgttttc tctgatggcg attcagttct tcagttcggg ggacctcctg aatctagctc | 2880 |
| tgtcaccgag tttgaccgga caaaagatac tccggcggtc gacacccttg tcaacttgac | 2940 |
| gactccaaac gaggcccccc ctgtaaccga ttctcgtgaa cttgccgaac tcaaacgccc | 3000 |
| gcgtttttcc gcacaagccc taattgaccg aggcggtcca ctcgctgatc tccatgcaga | 3060 |
| gataaaaaat cgggtatacg aacaatgcct ccaagcttgt gagcccggta gtcgtgcgac | 3120 |
| cccagccacc aagaagtggc ttgacaaaat gtgggatagg gtggacatga gaacttggcg | 3180 |
| ctgcacctcg cagttccaag ctggtcgtat tcttgcatcc ctcagtttcc ttcctgacat | 3240 |
| gatccgagac acaccacctc ctgtacccag gaagagccgg gttagtgaca gtgtcggtct | 3300 |
| gaagcaacta gtgactcagt gggataagaa actgagtgtg gccccccgag aagggcttat | 3360 |
| tgagtcagtg ctcgaccaaa ccgttccgcc gcccacggat gtccagcaag gagataccac | 3420 |
| ccctccccat gaaccacccg atgcgccgga tttgcctggt cgagtgggta caagcagagg | 3480 |
| ttggaagggt cttacgcttt ccggcgcccg cctcgcgggg tctgtcagcc agcgcctcat | 3540 |
| ggcatgggtt tttgaagttt actcccatct cccagctttt atgctcacac ttttctcgcc | 3600 |
| gcggggctct atggcttcag gtgattggct ttttgcaggt attgttctac ttgctctctt | 3660 |
| gctctgtcgc tcttacccaa tactcgggtg cctaccctta ttgggtgtct tttctggttc | 3720 |
| tctgcggcgc gttcgtctgg gcgtatttgg ctcttggatg gcttttgctg tatttttatt | 3780 |
| cacgactcca tccaacccag tcggttcttc ttgtgagcac gattcgccgg aatgtcacgc | 3840 |
| tgagcttctg gctcttgagc agcgccaact ttgggaacct gtgcggggcc ttgtggtggg | 3900 |
| cccctcaggt ctcctatgcg tcattcttgg taggttactc ggtgggtcac gttatctctg | 3960 |
| gcttgctttc ttacgtctat gcttgcttgc agatttggcc ttttctcttg tttatgtggt | 4020 |
| gtcccagggg cgttgccaca agtgctgggg aaagtgtata aggacagccc ccacggaggt | 4080 |
| ggctctcaat gtgttccctt tttcgcgcgc cacccgttcc tctcttgtgt ccttgtgtga | 4140 |
| tcgattccaa acaccaaaag gggttgatcc ggtgttcttg gcaacaggtt ggcgcgggtg | 4200 |
| ctggtgtggt gagagcccca ttcatcaatc acaccaaaaa cccatagctt acgccaactt | 4260 |
| ggatgaaaag aagatatctg cccaaacggt agtcgctgtt ccatacgatc ctaaccaagc | 4320 |
| tatcaaatgc ctgaaagttt tgcaggcggg gggggccatt gtggaccaac caacacctga | 4380 |
| ggtcgttcgt gtatccgaga tccccttttc agctccgttt ttcccgaaag ttccggtcaa | 4440 |
| cccagactgc aaggtagtgg tagattcgga cacttttgtg gctgcggttc gttgcggtta | 4500 |
| ctcaacaaca caactggttt tgggtcgggg caattttgcc aagttgaatc aggccccttc | 4560 |
| taaggcctct gcttacacaa aaacgactgg tggggcctct tatacttttg ctgtagttca | 4620 |
| agtgtctgtg tggactctta tccacttcat tctcggcctt tggttgatgt cgcctcaagt | 4680 |
| ttgtggtcga ggaacttccg acccatggtg ttcagatcct ttttcgtacc ccacttatgg | 4740 |
| cccaggcgtt gtgtgctcgt ctcaactttg tgtatccgcc gatggtgtta ccctaccgtt | 4800 |
| gttttcggcc gtggcccgac tttctggcag ggaggtgggg atttttattt tagtgtttgt | 4860 |
| ctccttggct gctttagccc atcgctgggc ccttaaggct gacatgttag taatcttttt | 4920 |
| agcgttttgt gcttacgcat ggcccatgag ttcctggcta atttgccttt tcccaacact | 4980 |
| cttaaggtgg atcaccctcc accctctcac catactttgg gtgcattcat tcttagtgtt | 5040 |
| ctgcctgccg gctgccggcg ttctttcatt agggataact ggtcttctct gggcagttgg | 5100 |
| acgctttacc caagttgccg gacttatcac accttatgac atccaccaat atacttctgg | 5160 |
| gccgcgtggt gcaactgctg tggccacggc tccagagggc acttacatgg ccgccgtccg | 5220 |
| gagagctgct ctaactgggc gaactctaat cttcaccccg tctgcggtcg ggtcccttct | 5280 |
| cgaaggtgct ttcaggactc ataaaccttg cctcaacacc gtgaatgttg tgggttcttc | 5340 |
| cctcggttct ggaggagtct tcaccattga tggcaggaaa actgtcgtca ctgccaccca | 5400 |
| cgtgctgaat ggcgacacag ctagagttac cggtgactcc tacaaccgca tgctcacttt | 5460 |
| caagaccaat ggtgattatg cctggtccca tgctgatgac tggcagggtg ctgccccagt | 5520 |
| ggttaagatt acaaaaggat accgcggtcg tgcttattgg caaacatcaa ccggtgtcga | 5580 |
| gcccggtatc attggagagg ggttcgcctt ctgttttacc agctgcggtg actcggggtc | 5640 |
| gcctgttata tctgaagctg gtgaccttat cggcatccat actggttcaa acaaacttgg | 5700 |
| ttcaggtctt gtgacaaccc ctgaagggga gacctgctca attaaggaaa ctagactttc | 5760 |
| tgacctctct aagtattttg cgggtccgtg cgtccctctt ggggacatta agttaagccc | 5820 |
| tgccatcatt cctgacatga catctgttcc aagcgacttg gcatcgcttc ttgcttctgt | 5880 |
| ccctgtcatg gagggcggtc tctcgactgt tcaacttttg tgtgtctttt ttcttctctg | 5940 |
| gcgcatgatg ggtcatgctt ggacacccat cgttgctgtg ggcttctttt tgctgaatga | 6000 |
| aatccttcca gcagttttag tccgagccgt gttctctttt gcactctttg tgcttgcatg | 6060 |
| ggccaccccc tggtctgcac aggtgcttat gatcagactt cttacagcgt cccttaaccg | 6120 |
| gaacaagtct tctctggcgt tttacgcatt cgggggtgtc gtcggcctgg ctgctgaaat | 6180 |
| cggaactttt gctggtaaac tacctgaatt gtctcaagct ctttcgacat actgcttttt | 6240 |
| gccaagattt cttgctgtat ctagttgtgt tcccatcatc atcatcggtg ggcttcatgt | 6300 |
| tctcggcgtg attttgtggc tattcaaata ccggtacctt catgacgtgc tggttggtga | 6360 |
| tgggagtttt tcaaaagcct tcttcctacg gtattttgct gagggcaatc tcagaaaggg | 6420 |
| tgtttcacaa tcctgtggca tgagtaacga gtccctaacg gctgctttgg cctgcaagtt | 6480 |
| gtcgcaagct gaccttgaat ttttatccag cttaacgaac ttcaagtgct ttgtgtctgc | 6540 |
| ctctaacatg aaaaatgctg ctggccagta cattgaagca gcgtatgcca aggccctgcg | 6600 |
| ccaagagttg gcctctctag ttcaggttga caaaatgaaa ggagttttgg ccaagcttga | 6660 |
| ggcctttgct gaaacagcca ccccgtctct agacacgggt gatgtgattg ttctgcttgg | 6720 |
| gcaacaccct cacggatccg tcctcgacat aaatgtgggg actgagagga aaactgtgtc | 6780 |
| cgtgcaagag acccggagtt tgggcggctc caggtttagt gtttgtactg ttgtgtccaa | 6840 |
| tacccctgtg gatgccttaa ccgacatccc acttcaaaca ccaacccccc tttttgagaa | 6900 |
| tggtccacgc catcgtagcg acgaagacga tcttaaggtt gagaggaaaa agaaacactg | 6960 |
| tgtgtccctc ggcttccaca acatcaacgg taaggtttac tgtaaaattt gggacaagtc | 7020 |
| caccggtgat accttttaca cagatgattc ccggtatact caagaccatg tttttcagga | 7080 |
| caggtcagcc gactacaggg acagggacta tgaaggtgtg caagccaccc ccccacaggg | 7140 |
| atttgatcca aaatctgaaa ccccggttgg cactgtcgtg atcggcggta tcacgtataa | 7200 |
| caggtatctg gtaaaaggta gagaggttct ggttctcaag cccgacaact gtcttgaagc | 7260 |
| cgccaggttg tctcttgagc aagctctcgc tgggatgggc caaacttgtg acctcacggc | 7320 |
| cgctgaagtg gaaaagctaa agcgtatcat cagtcaactt caaggcttga ccactgaaca | 7380 |
| agctttaaac tgttagccgc cagcggcttg acccgctgtg gccgcggcgg cttagttgtg | 7440 |
| actgaaacgg cggtgaaaat cataaaatac cacaacagaa ctttcacctt aggcccttta | 7500 |
| gacttgaaag tcacatccga ggtggaggtg aagaaatcaa ctgagcaagg ccacgctgtt | 7560 |
| gtggcaaact tatgttccgg tgttgtcttg atgagacctc acccaccgtc ccttgttgac | 7620 |
| gttctcttga aacccggact tgacacgaca cctggtattc aaccggggca tggggccggg | 7680 |
| aatatgggcg tggacggttc tatttgggat tttgaaaccg cacctacaaa ggcggaactt | 7740 |
| gagttgtcca agcaaataat tcaagcatgt gaagtcaggc gcggggacgc cccgaacctc | 7800 |
| caactccctt ataagctcta ccctgttaga ggggatcctg aacggcataa gggtcgcctt | 7860 |
| atcaatacca ggttcggaga tttgccctat aaaactcctc aggacaccaa gtccgcgatc | 7920 |
| catgcggctt gttgcttgca ccccaatgga gcccccgtgt ctgatggcaa atccacgcta | 7980 |
| ggcaccactc ttcaacatgg ttttgagctt tatgtcccca ctgtgcccta tagtgtcatg | 8040 |
| gagtaccttg attcacgccc tgacacccct ctcatgctca gcaaacatgg tacttccaag | 8100 |
| gctgctgcag aagatctcca aaaatatgat ctgtccaccc aaggatttgt cctgcctggg | 8160 |
| gttctgcgcc tagtgcgcaa attcatcttc ggccacatag gtaaggcgcc gccattgttc | 8220 |
| cttccatcaa cctatcccgc taagaattct atggcaggga tcaacggtca gaggtttcca | 8280 |
| acaaaggatg tccagagcat acctgaaatt gatgagatgt gtgcccgtgc cgtcaaggag | 8340 |
| aattggcaaa ctgtgacacc ttgtaccctc aagaagcagt actgttccaa gcctaaaacc | 8400 |
| aggaccatcc tgggcaccaa caatttcatt gccctggctc acagatcagc actcagcggc | 8460 |
| gtcacccagg catttatgaa gaaggcttgg gagtccccaa ttgctttggg aaaaaataaa | 8520 |
| ttcaaagagc tgcattgcac ggttgccggc aggtgccttg aggccgatct ggcctcctgt | 8580 |
| gatcgcagca cccccgccat tgtaagatgg tttactgcca acctcctgta tgaacttgca | 8640 |
| ggatgtgaag accatttgcc cagctatgtg cttaactgct gtcatgacct cgtggcaaca | 8700 |
| caagatggcg ccttcacgaa acgtggtggc ctgtcgtccg gggatcccgt cactagtgtg | 8760 |
| tccaacaccg tgtattcact agtgatttat gcccagcaca tggtattgtc agccctgaaa | 8820 |
| atgggccacg agattggtct caagttcctc gaggaacagc ttagattcga ggaccttctt | 8880 |
| gaaattcagc ccttgctggt atactctgac gaccttgtct tgtatgctga aaaacccact | 8940 |
| tttcccaatt accattggtg ggttgaacat ctcgacttga tgttgggttt caagacggac | 9000 |
| ccgaagaaaa ctattataac agacaagccc agcttccttg gctgcagaat tgaggcaggg | 9060 |
| cgacaactag tccccaatcg tgaccgcatt ctcgccgctc ttgcatacca catgaaggca | 9120 |
| cagaacgttt cagagtatta tgcatctgct gctgcagttc ttatggattc atgtgcctgc | 9180 |
| atcgaccatg accctgagtg gtatgaggac ctcatctgcg gcatcgccag gtgcgctcgt | 9240 |
| caagatggct atagttttcc cggcccggca ttttttatgt ccatgtggga gagactgaaa | 9300 |
| agccacaatg agggaaagaa attccgccac tgcggcatct gtgatgccaa ggccgaccac | 9360 |
| gcgtccgcct gtggacttga cttgtgtctg tttcactcat attttcacca gcactgccca | 9420 |
| gtcactctgg gttgtggtca ctatgccggt tcaaaggaat gccagcagtg tcagtcacct | 9480 |
| attggaaccg gcaagtctcc tcttgacact gtgctgaaac aaatcccgta taaacctcct | 9540 |
| cgcactgtca tcatgagggt ggacaacaag acaacggccc tcgatccagg gagatatcag | 9600 |
| tcccgtcgag gcctcgttgc agtcagaaga ggcattgcag gcaatgaagt cgatcttgct | 9660 |
| gatggagact accaggtagt gccccttttg ccgacttgca aagacataaa tatggtgaag | 9720 |
| gtcgccagta atgtgctagt tagcaagttc atagtgggac cgccaggttc cggaaagacc | 9780 |
| acctggttat tgagtcaggt ccaggatgaa gatgtcattt acacacccac tcatcagacc | 9840 |
| atgtttgaca tagtcagtgc tctcaaagtt tgcaggtatt ccataccagg ggcctcagga | 9900 |
| ctcccttttc caccgcctgc caggtccggg ccgtgggtta agctcattgc cagcgggcac | 9960 |
| gtccccggtc gagtgtcgta cctcgatgag gccggatatt gcaatcattt ggatatactt | 10020 |
| agactacttt ctaaaacacc tctcgtgtgc ttgggtgacc ttcagcaact tcaccctgtc | 10080 |
| gggttcgatt cccactgtta tgtttttgat caaatgcctc aggagcagct gaccactatt | 10140 |
| tatagatttg gtcccaacat ctgcacagcc atccagcctt gctacagaga aaaacttgaa | 10200 |
| tccaaggcta ggaacaccag ggtggttttc accacccggc cagtgacctt tggtcaggtg | 10260 |
| ttaacaccgt accataaaga tcgcgttggc tctgcgataa caatagattc atcccaaggg | 10320 |
| gccacctttg atgttgtgac attacacttg ccatctccga aatccctaaa taaatcccga | 10380 |
| gcacttgtgg ccatcactcg ggcgagacat gggttgttca tttacgaccc ccacaaccaa | 10440 |
| ctccaggagt tttttaacct gactcctgag cacactgatt gtaacctagt gttcagccgt | 10500 |
| ggggacgagc tggtggtttt gagtgcggat aatacagtca caactgtagc gaaggcccta | 10560 |
| gaggtgggtc catctcgctt ccgagtgtca gacccgaggt gcaagtctct tttggctgcc | 10620 |
| tgttcggcta gtctggaggg gagctgcatg ccgctaccac aagtggcaca caacctgggg | 10680 |
| ttttactttt ccccggacag ttcagcattt gcacctctgc cagaagagtt ggcgccacat | 10740 |
| tggccagtgg ttacccacca gaacaattgg gcgtggcctg accggcttgt tgccagcatg | 10800 |
| cgcccgattg atgcccgcta cagcaaacca atggtcggtg cagggtatgt agtcgggccg | 10860 |
| tccacctttc tcggcacccc cggtgtggtg tcatattatc tcacactata cgtcaaaggt | 10920 |
| gagcctcagg ccttaccaga aacacttgtt tcaacaggac gtatagccac agactgtcgg | 10980 |
| gagtatctcg acacggctga agaagaggca gcaagggaac tcccccacgc attcattggc | 11040 |
| gatgtcaaag gtaccacgat tgggggatgt catcacatca catcaaaata tttgcccagg | 11100 |
| ttcctgccca aggactctgt tgccgtagta ggagtgagtt cgcctggtag agctgctaaa | 11160 |
| gccgtgtgca ctctcaccga tgtgtatctt cccgaactcc gaccatatct gcaacctgaa | 11220 |
| acggcatcaa aatgttggaa actcaagtta gatttcaggg acgttcgatt aatggtctgg | 11280 |
| aaaggagcta ccgcctactt ccagttggaa gggcttacat ggtcagcgtt gcccgattat | 11340 |
| gccaggttta ttcagctgcc caaggacgcc gtggtataca tcgacccatg cataggaccg | 11400 |
| gcgactgcca accgcaaggt tgtgcgaacc acggattggc gggccgacct ggcagtgaca | 11460 |
| ccgtatgatt acggggccca gcacattctg acaacagctt ggtttgagga cctcgggccg | 11520 |
| cagtggaaaa ttttggggtt gcagcccttc aggcgagcgc ttggccttga aaacaccgag | 11580 |
| gactgggcga ttcttgcgcg ccgtatgaat gacggcaagg attacattga ctacaattgg | 11640 |
| cattgcgtcc gaggacgccc acgcgctatc tacgggcgcg ctcgtgacca tacttaccat | 11700 |
| tttgccttgg gcacagaatt gcaggtggag ctgggtaaac cccaactgcc gcctgagctg | 11760 |
| gtaccgtgaa cctgaagtga tgcaatgggg ttgttatgga gtaaaatcag ccagctgttt | 11820 |
| gtggacgcct tcacagagtt ccttgttagt gtggttgata tcgtcatctt tcttgccata | 11880 |
| ctgtttgggt tcaccgtcgc agggtggtta ctggtctttt ttctcagatt ggtttgctcc | 11940 |
| gcgattctcc gttcgcgctc tgccattcac tctcccgaac tatcgaaggt cctatgaagg | 12000 |
| cctgctaccc aattgcaggc ctgatgtccc acaattcgca ttcaagcacc cattgggtat | 12060 |
| gctttggcac atgcgagttt cccaattgat tgacgagatg gtctctcgtc gtgtctacca | 12120 |
| gaccatggaa caatcaggtc aagcggcctg gaagcaggta gttggtgagg ccacccttac | 12180 |
| gaagctatca aggctcgatg tagttaccca cttccagcac ttggccgcaa cagaggcgga | 12240 |
| ttcttgccgc tttcttagct cacgactcgt gatgctaaag aatcttgccg ttggtaatgt | 12300 |
| gagcctacag tacaacacca cgtcagacca cgttgaactc atttttccca ctccaggtgc | 12360 |
| gaggcccaag ttgaccgatt tcagacaatg gctgatcagt gtccatgctt ctattttttc | 12420 |
| ctctgtggcc tcatctgtta ccttgtttgt ggtgctttgg cttcgagtcc caatgctacg | 12480 |
| ctatgctttt ggtttccatt ggctcacggc aacacatcat tcgagttaac tattaattac | 12540 |
| accatatgca agccctgcct caccagtcaa gcggctaaac aaaggcttga acctggtcat | 12600 |
| agcatgtggt gcaggatagg ggacaccagt tgtgaggaga gtgaccacga tgagttgtca | 12660 |
| atgaccatcc cgtctgggta cgataacctc aaactcgagg gctattatgc ttggctggcc | 12720 |
| ttcctgtcct tttcctacgc ggcccaattc catccggagc tgtttggaat agggaacgtg | 12780 |
| tcgcgtgttt ttgtggacaa gcgacaccag ttcatttgtg cggagcatga tggacccaat | 12840 |
| tcaaccgtgt ccattaatca taacatctcc gcatcgtacg cggtgtatta ccatcatcag | 12900 |
| gtagacggag gtaactggtt ccacttggaa tggctgcggc cgttcttctc ctcctggttg | 12960 |
| gtgctcaatg tctcatggtt tctgaggcgt tcgcctgcaa gccctgtttc tcgacgcatc | 13020 |
| tatcagatat taagaccaac acgaccgcgg ctgccggttt tatggtcctt caaaacattg | 13080 |
| aatgtctcca acctcacacg ggccccgcag cgcaagggac catcccccaa gcgaaacggt | 13140 |
| cacaatgtcg ccaagccgtc ggcactcccc agtacatcac gataacggct aacgtgactg | 13200 |
| acgaatcata cttgtataac gcagatttgc taatgctttc tgcgtgcctt ttctatgcct | 13260 |
| cagaaatgag cgagaaaggc tttaaagtca tctttgggaa cgtctctggc gtcgtttccg | 13320 |
| cttgtgtcaa tttcacggat tatgtggctc atgtgaccca acatacccag cagcaccatc | 13380 |
| tggtaattga ccacgtccga ttactgcatt tcttgtctcc atccacaatg aggtgggcta | 13440 |
| caaccattgc ttgtttggtc gccattctcc tggcgatatg aaatgttctc acagattggg | 13500 |
| gtgtttcttg actccgcact cctgcttttg gtggtttttt ttgctgtgta ccggcttgtc | 13560 |
| ctggtccttt gtcgatggca acggcaacag ctcgacatac caatacatat ataacttgac | 13620 |
| gatatgcgag ctgaacggga ccgcctggtt gtccagccac ttttcttggg cagtcgagac | 13680 |
| ctttgtgctt tacccagtcg tgactcatat tctctcactg ggttttctca ccacaagcca | 13740 |
| tttttttgac gcgctcggtc tcggtgctgt gtccatcaca ggtttttttg gcaaacggta | 13800 |
| cgtactcagc agcatctacg gtgcttgtgc tctcgcagcg ttcgtgtgct ttgccatccg | 13860 |
| tgctgctaaa aattgcatgg cttgccgcta cgcccgcacc cggttcacta acttcattgt | 13920 |
| agacaaccgg gggaggatcc atcggtggag gtctccaata gtggtggaga aattgggtaa | 13980 |
| agctgaaatt ggcagcgacc ttgtcaccat caaacatgtc atcctcgaag gggttaaagc | 14040 |
| tcaacccttg acaaggactt ctgctgagca atgggaagcc tagatggttt ttgtgatgag | 14100 |
| cctcccgctg cgcaaaatct tgtgctagcc tttagcatta catacacacc tgtaatgata | 14160 |
| tatgccctta aggtgtcacg cggtcgactc ctagggctgt tgcacatctt gatattcctg | 14220 |
| aactgctctt tcactttcgg gtatatgacg tatgtgcatt ttcagtctgc caaccgtgtt | 14280 |
| gcactcactt tgggggccgt tgttgccctc ctgtggggcg tttacagctt cacagaatca | 14340 |
| tggaagtttg ttacttccag atgcagattg tgctgcctag gccggcggta cattctggcc | 14400 |
| cctgcccacc acgtagaaag tgctgcaggt ctccactcaa tcccagcgtc tggtaaccgc | 14460 |
| gcatacgctg tgagaaagcc cggactaaca tcagtgaacg gcactctagt accaggactt | 14520 |
| cggagcctcg tgctgggcgg caaacgagct gttaaacgag gagtggttaa cctcgttaag | 14580 |
| tatggccggt agaaaccagg gccagaagaa aaagaaaagt acagctccaa tggggaatgg | 14640 |
| ccagtcagtc aatcaactgt gccagttgct gggcacaatg atgaagtccc agcgccagcg | 14700 |
| acctagggct ggacagacta aaaggaaaaa gtctgagaag ccacattttc ccttggctgc | 14760 |
| tgaagatgat attcggcacc acctcaccca gactgaacgc tccctctgct tgcaatcgat | 14820 |
| ccagactgct ttcaaccaag gcgcaggaac tgcgtcgctt tcatccagtg ggaaggtcag | 14880 |
| ctttcaggtc gagtttatgt tgccggttgc tcatacagtg cgcttaattc gcgtgacttc | 14940 |
| tacatccgct agtcaggatg caagttaatt cgacagtcag gtgaatggcc gcgattggcg | 15000 |
| tgtggccttt gagtcaccta ttcaattagg gcgatcacat gggggtcata cttattaggc | 15060 |
| aagatccatg tgaccgaaat t | 15081 |

The cDNA consensus sequence of PRRS strain ES13-49 at P85 has been assigned GenBank Accession number MK024325 (SEQ. ID. NO: 2). The cDNA consensus sequence designated SEQ. ID. NO: 2 is:

| | |
|---|---|
| atgatgtgta ggggagatac cctacacaca caacactcct ggtgtttgtg tgccttggag | 60 |
| gcgtgggtac agccccgccc cacctcttgg cccctgttct agcccaacag gtatccttct | 120 |
| ccctcggggc gagtgcgccg cctgctgctc tcttgcagtg ggaaggacct cccgagtatt | 180 |
| tccggagagc acctgcttta cgggatctcc accctttaac catgtctggg acgttctccc | 240 |
| ggtgcatgtg caccccggct gcccgggtat tttggaacgc cggccaagtc tattgcacac | 300 |
| ggtgtctcag tgcgcggcct cttctctctc cagagcttca agacactgat ctcgctgcaa | 360 |
| ttggcttgtt ttacaagcca agaaacaagc ttcactggaa ggtccctatt ggcattcctc | 420 |
| aagtggagtg caccccatct gggtgctgct ggctctcagc catctttccc atagcgcgca | 480 |
| tgacctccgg caaccacaat ttcacccaac gactcataaa ggttgccgat gtgttgtacc | 540 |
| gtgatggttg tttgactcgt caacaccttc gtgaacttca agtttatgag cgcggctgca | 600 |
| attggtaccc gattacgggg cctgtgcccg gagtggctgt gtatgcgaac tccatgcacg | 660 |
| tgtccgacca gccgttccct ggtaccaccc atgtgttaac gaacttgcct ttacctcaac | 720 |
| aggcctgtcg gcagccgttc tgtccatttg aggaggctca ttctaacgtg tataggtgga | 780 |
| atggactcgc gatttttgtg gattccactt ccgacggccg gtcccgcatg atgtggacac | 840 |
| cggggtctag cgactcgact gccttagaag tgctaccacc tggactagga cgtcaagccg | 900 |
| aaatcctcac ccggagtttt cctgcccacc accctgttaa cctcgctgac tgggagctca | 960 |
| ccgagacccc tgaatccggt ttctccttca gcatgtctca gtcttgtggt taccttgccc | 1020 |
| aaaaccctga cgtttttgat ggcaagtgct ggctttcctg tttttttgac ctgccgactg | 1080 |
| aggtatggcg tcgtgaggag catctggcta gtgccttcgg ttatcaaact aaatggggcg | 1140 |
| tgcatggcaa atacctccag cgcagacttc aaatcaatgg agttcgcgct gtagtcgatc | 1200 |
| ctgatggtcc tatccatgtt gaagcgttgt cttgccccca atcttggatc agacacctga | 1260 |
| ctctagacgg tgacgtgacc ccaggattcg ttcgcctgat gtctctccgt attgtaccga | 1320 |
| acacagaacc ggccactctc ccggtctttc ggtttggagc gcataaatgg tatggcgctg | 1380 |
| ccggcaaacg agcccgtgct aggcgtgccg ccagaaatgg gaaggactca gccactgccc | 1440 |
| ccacggccac ccaactgatc cctgcctgtg gaacaaccac ttattccccg ccaacagacg | 1500 |
| ggtcttgtgg ctggcatgtt ctcgccgcca tagttaatcg gatgatgcat aatgatttta | 1560 |
| catctcctct gactcagtat aacagaccac aggacgattg ggcgtccgat tatgaccttg | 1620 |
| ctcaggcaat ccagtgtatg cgactgcctg ctactatagt tcgtggtcgt gcctgcccta | 1680 |
| acgccaagta ccttataaaa ctcaatggag tccattggga ggtagaggtg aggtctggaa | 1740 |
| tggctccgcg tcttctttct cgtgagtgca ttgttggcgt ctgctctgaa ggctgtatcg | 1800 |
| caacgcctta ccctgaaggc gagctacccg agcgtgcact agaggccttg gcggctgctt | 1860 |
| acagactacc ttccgactgt gtaagttctg gcattgccga cttccttgct gacccacctc | 1920 |
| ctcaggaatt ctggaccctc gacaagatgc taacctcccc gtcaccggag cggtccggtt | 1980 |
| tctccagttt gtataaatta ctattagagg ttgttccaca aaagtgcgga gctacggagg | 2040 |
| gggctttcgt ctatgctgtt gagaggatgt tgaaggactg cccaagctcc aaacaggcaa | 2100 |
| tggccctcct ggctaaagtc aaagtcccgt cttcgaaggc cccgtctgtg tccttggacg | 2160 |
| agtgtttccc cacggacgtt ccagccgacc ccgagccaac atctcaggaa aagcctcaaa | 2220 |
| gttctggcac taccacagtc ctgtgttcgc cgaatacaaa agagtctgag gaggtggcct | 2280 |
| tgcaaggcgt tcaggagagc agccacaagg ccgcccactc tgcagtcctt gttgaggaac | 2340 |
| ttagcgggaa gcgggcgcag gaggttgccg gcgagctaca ggagttcggc gactgtggct | 2400 |
| tggtaatcgg gagtgctcaa gacggcattc tggaggatga gccattggac ttgtcccgat | 2460 |
| cagcgttggg ggccacaacg attctcgtga gaaaccaaac acccaacaat tcgggttttg | 2520 |
| gcactggtac tcctcctgcc actgttcaag agcccgtctt tacagggctc atgtcttatt | 2580 |
| gcgttgagca ttgtaaaacg gagtccgata acagcagttt acctctggat ctgtctaatg | 2640 |
| cgcaaacctc ggaccagcct ttaaatctac ccttggctgc ttggccagtg agaaccaccg | 2700 |
| catctgaccc tggctgggcc cacggtaggc gtgtgcctgt ctttgtaaag ccccggggca | 2760 |
| ctctctccga tggcgattca gtccttctgt ttggggggct ttccgaatcc agctctgtta | 2820 |
| tcgagtttga ccaatcgaaa gacgtcccag tgaccgatgc ccccgtcgac ttgaccaccg | 2880 |
| cgaacggagc cctctctggg atcgactccc ttgaatttgc tgaactcaag cgcccgcgct | 2940 |
| actccgctca agctttgatt gaccgaggtg gtccactagc cgatgttcat gcaaagataa | 3000 |
| agagccgggt atatgaacaa tgccttcagg cttgtgagcc cggcagtcgt gcaaccccag | 3060 |
| ccaccaggga ttggctcaat aaaatgtggg aaagggttga tatgaagact tggcgctgta | 3120 |
| cttcgcagta ccaagctggt cacattctcg cgtctcttaa atttcttcct gacatgatcc | 3180 |
| aagacacgcc acctcctgtt cccaggaaga accgagctag tgatcatgcc gaaccaaaac | 3240 |
| gtctggtggc gcagtgggac aagaaattga gtgtggtctc ttccccaaaa ccggttgagc | 3300 |
| cagcgcctga ccggaccacc cctttgcctg cggacatcca gcaagagggt gttgcctcct | 3360 |
| ccgacagatt aacccgtgcg ccagacctcc ctagtcaagt gagcacgggc gggagttgga | 3420 |
| aagaccgcat gcttttcggc gctcgtttcg cggagtccat tggtcagcgc atcacagcac | 3480 |
| gggtttttga aactttctcc catctcccag cttttgtgct cgcacttttc tcgccgcggg | 3540 |
| gcgctatggc ttcaggtgat tggctgtttg caggtattgt tttacttgct ctcctgctct | 3600 |
| gtcgccctta cccagtactc gggtgcttac ccttactggg tgtcttttct gggtctgtgc | 3660 |
| ggcgtgttcg tctgggtgtt tttggttctt ggatggcttt tgctgtattt ttattctcga | 3720 |
| ctccacccga cccagtcggt tcttcttgcg gccacgattc gccggagtgt catgctgagc | 3780 |
| ttttggctct tgagcagcgc caactttggg aacctgtgcg cagccttgtt gttggcccct | 3840 |
| cgggtctcac gtgcgtcatt tttggtaggt tactcggtgg gtcacgttat ctctggcata | 3900 |
| ttctcctacg tttatgcatg cttgcagatt tggccctttc tcttatttat gtggtgtccc | 3960 |
| aagggcgttg tcacaagtgt tggggacagt gtataagaac agctcctgca gaagtggctc | 4020 |
| ttaacgtctt cccttttttg cgtgccaccc gtgcctctct cgtgtccgtg tgcgaccgat | 4080 |
| tccaatcgcc aaaaggtgtt gatcctgtgc acttggcaac cggctggcgt gggtgttggc | 4140 |
| gtggtgagag tcctatccac caaccacacc aaaagcccat agcttatgcc aatttagatg | 4200 |
| aaaagaaaat atccgccaaa acggtggtcg ctgtcccata tgatcccagt caggccatca | 4260 |
| aatgcctaaa agttctacag gcgggaggag ctattgtaga ccagcccacg ccagaggtcg | 4320 |
| ttcgcgtgtc tgaaatccct ttctcagccc catttttccc aaatgttccg gtcaacccgg | 4380 |
| attgcagggt tgttgtagac tcggacactt ttgtggcagc agtccgctgt ggttactcga | 4440 |
| cggcacaatt ggttttgggc caaggcaact tcgccaaatt aaaccaaatc ccccttggga | 4500 |
| gttccacctc taccagaacg actggcgggg cttcttacac ccttgctgtg gctcaagtgt | 4560 |
| ctgtgtggac cctcgttcat ttcattcttg gtctttggtt cacatcgcct caagtgtgtg | 4620 |
| gtcgggggac ctccgacccg tggtgttcaa atcctttttc atatcccact tatggccccg | 4680 |
| gagttgtttg ttcctcacga ctctgtgtgt ctgccgacgg ggtcactcta ccattgtttt | 4740 |
| cagccgtggc gcagctctcc ggtagagagg tgggaatttt tgttctggtg ctcgtctcct | 4800 |
| tgattgctct agctcatcgt atggctctta aggcagatat gttagtggtc tttttggctt | 4860 |
| ttgggggtta cgcctggcct atgagctctt ggttaatctg tttctttcct ttactcctga | 4920 |
| agtggatcac tcttcaccct ctcaccatgc tttgggtgca ctcgttttta gtgttttgtc | 4980 |
| tgcccgcagc cggcgtcctc tcgctgggga taactggcct cctttgggtg actggccgtt | 5040 |
| ttacccaggt agccggaatc atcacacctt atgacatcca tcagtacacc tctgggccgc | 5100 |
| gtggtgctgc tgctgtggcc accgccccag aaggcactta catggccgcc gttcggagag | 5160 |
| ctgccctaac cgggcgaacc ttaatcttca ctccgtccgc agtcgggtct cttcttgaag | 5220 |
| gtgctttcag gactcgcaaa ccctgcccta ataccgtgaa tgttgttggt tcttccctcg | 5280 |
| gttccggagg agttttcacc attaatggaa agaaaatcgt cgtcaccgct acccatgtgt | 5340 |
| tgaacggcga tgcagccaga gtcactggtg actcttacaa ccgcatgcac actttcaaga | 5400 |
| ccaatggtga ttacgcctgg tctgatgcag acaactggca aggtgttgcc cccatggtca | 5460 |
| aggttgcaaa ggggtatcgc ggtcgtgcct actggcaaac atcaactggt gttgaacccg | 5520 |
| gtattgtcgg ggaaggtttc gccttttgct tcactaactg cggtgactcg gggtcacccg | 5580 |
| tcatttcaga gtccggtgac ctcatcggga tccataccgg ttcaaataaa ctgggctctg | 5640 |
| gccttgtgac aacccctaac ggggagacct gttccatcaa agaaactaaa ctctctgatc | 5700 |
| tttccaagta ttttgctggc ccgagtgtcc ctctcgggga caccaagttg agcccgacca | 5760 |
| ttatccctga tgtgacatcc attccgagtg acttagcatc gctcctagct tccgtccctg | 5820 |
| taatggaagg tggtctttcg accgttcaac tcttgtgtgt cttctttctc ctctggcgta | 5880 |
| tgatgggtca tgcctggaca cctgtcgttg ccgtgggttt cttcttgcta aatgaaattc | 5940 |
| ttccagcagt tttagttcgt gccgtgtttt ccttcgcact ctttgtgctc gcgtggctta | 6000 |
| ccccttggtc cgcacaggtg ctaatgatta gactcctcac agcgtccctt aaccgcaaca | 6060 |
| agctttcctt agcgttctac gcactcgggg gcatcgtcgg tttggctgct gaaattggga | 6120 |
| ctttcgctgg cagactgcct gatctgtctc aagctctttc gacgtactgc ttcctgccta | 6180 |
| gggtcattgc tgtgaccagt tgtgttccca tcatcatcat tggcgggctt catgctctcg | 6240 |
| gtgtgatctt gtggttgttc aaatatcggc acctccacgc catgttggtt ggtgacgggg | 6300 |
| ctttctcaag tgcattcttc ctgcggtatt ttgcagaggg taaccttagg aagggggttt | 6360 |
| cgcagtcctg tggcatgagc aacgaatccc taacggctgc cttagcttgt aagttatcac | 6420 |
| aggctgacct agatttcctg tcaagcttga cgaacttcaa atgctttgtg tctgcttcaa | 6480 |
| acatgaagaa tgctgctggc caatatatcg aagcagcata tgccaaagct ctgcgccgag | 6540 |
| agctggcctc cctagtccag gtcgacaaaa tgaaaggagt cttgtccaag ttggaagctt | 6600 |
| ttgctgagac ggccaccccg tcccttgaca caggtgacgt ggttgtgctg ctcgggcaac | 6660 |
| atccccatgg atctatcctt gacatcaatg tggggactga aagaaaaact gtgtccgtgc | 6720 |
| aagagactcg gaacttgggc ggctccaagt tcagtgtctg tactgttgtg tccaacacac | 6780 |
| ctgtggacgc cttgaccgat gttccgcttc aaacaccgac tccgctcttc gagaacggcc | 6840 |
| cgcgtcaccg ctgtgaggaa gacgatctta aagtcgagag aatgaggaaa cattgtgtgt | 6900 |
| ctctcggctt ccacaatatc aatggcaaaa tttattgcaa agtctgggat aagtccactg | 6960 |
| gtgacacctt ttatacggat gattcccggt atacccaaga ctatgctttt caggacaggt | 7020 |
| cagccgacta cagagaccgg gactatgagg gtgtgcaagt cgcctctcaa caaggattcg | 7080 |
| acccaaagtc tgaaactcct gttggcactg tcatgatcgg cggcatcatg tataacaaat | 7140 |
| acctagttaa aggcagggaa atcttggtcc ttaaacctga caactgccta gaagccgcca | 7200 |
| ggctgtccct tgaacaggcc cttgctggga tgggccagac ttgtgatctc acagccaccg | 7260 |
| aagtggaaaa gctaaagcgc atcattagtc aactccaagg tctgaccact gaacaagctt | 7320 |
| taaactgtta gccgccagcg gcttgacccg ctgtggccgc ggcggcttgg ttgtaactga | 7380 |
| aacggcggta aaaattgtaa aataccacag cagaactttc actttaggcc ctttagactt | 7440 |
| aaaagtcgct tctgaagtgg aagtaaagaa atcaactgag cagggccatg ctgttgtagc | 7500 |
| aaatttaagt tctggtgtcg tcttgatgag acctcaccca ccgtcccttg tcgatgtcct | 7560 |
| cctgaaaccc ggacttgaca caacacccgg cattcaacca gggcatggag ccgggaacat | 7620 |
| gggtgttgac ggttctattt gggattttga aactgcaccc acaaaggcag aacttgaatt | 7680 |
| gtccaaacaa ataattcaag catgtgaggt cagacgcgga gacgccccga acctacaact | 7740 |
| cccttacaag ctctatcctg ttagggggga ccctgagcgc catgccggtc gtctcaccaa | 7800 |
| taccaggttt ggagatttgc cttacaagac tccaggagac accaagtctg ccatccatgc | 7860 |
| ggcttgttgt ctgcacccca acggggtccc cgtgtctgat ggcaagtcca cactaggcac | 7920 |
| tacccttcaa catggttttg agctttatgt tcccacagtg ccctatagtg ttatggagta | 7980 |
| ccttgattca cgctctgata cccctcctat gttcactaaa catggcactt ctaaggctgc | 8040 |
| tgcagaagac ctccaaaaat atgatttatc cacccaagga tttgtcctgc ctggggtcct | 8100 |
| acgccttgtg cgcagatttg tctttggcca catcgggaaa gcaccgccgt tgttcctccc | 8160 |
| gtccacttac cctgccaaga actctatggc agggatcaac ggccagagat tcccaacgaa | 8220 |
| agacgttcag agtatacctg aaattgatga aatgtgtgcc cgcgccgtta aggaaaattg | 8280 |
| gcagaccgtg acgccctgca ccctcaagaa acagttctgt tccaagccca aaaccaggac | 8340 |
| cattctgggc accaacaact ttattgccct ggctcaccga tcggcgctca gtggcgtcac | 8400 |
| ccaagcattt atgaagaagg cttggaagtc cccgattgcc ctggggaaaa acaaattcaa | 8460 |
| agagctacat tgcactgttg ctggcaggtg tcttgaggct gatttggcct cctgtgatcg | 8520 |
| tagcaccccg gccatcgtga ggtggtttgc tgccaacctc ctgtacgagc ttgcagggtg | 8580 |
| tgaagagtac ttgcctagct atgtactcaa ctgctgccac gacctcgtgg caacacagga | 8640 |
| tggtgccttc acaaaacgcg gtggtttgtc atccggtgac cctgttacca gtgtgtcaaa | 8700 |
| caccatatat tcactggtga tctatgccca gcacatggtt ttgtcagcct taaaaatggg | 8760 |
| tcatgagatt ggtcttaagt ttctcgagga acagctcaaa ttcgaagacc tcctcgaagt | 8820 |
| ccagcccatg ttagtgtact ctgacgacct agtcttgtac gccgaaaggc ccaccttccc | 8880 |
| taattaccac tggtgggtcg aacaccttga cctaatgctg ggtttcaaaa cggacccgaa | 8940 |
| gaaaactgta ataactgata agcccagctt cctcggctgt aaaattgaag cagggcggca | 9000 |
| gctagttccc aatcgcgacc gtatcctagc cgctcttgca taccacatga aggcgcaaaa | 9060 |
| cgcctcagaa tattatgcat ctgctgctgc gatcctaatg gattcgtgtg cttgcattga | 9120 |
| ccacgatcct gagtggtatg aggacctcat ttgtggtatt gcccggtgcg ctcgccaaga | 9180 |
| tggctatagt tttccaggcc cggcattttt catgtcgatg tgggaaaaac taaagagcca | 9240 |
| caacgaaggg aaaaaattcc gccactgcgg tatctgtgat gccatggccg atcatgcatc | 9300 |
| tgcctgtggg cttgatttgt gtttgtttca ttcgcatttt caccagcatt gtccagtcac | 9360 |
| tctgaactgc ggtcaccgtg ccggcgcaaa ggaatgtccg cagtgccagt cgccagttgg | 9420 |
| ggttagcaaa tcccctctcg acactgtgct agaacaaatt ccatacaaac cccctcgtac | 9480 |
| tgtcattatg aaggtgagtg atagaacgac tgtcctcgac ccgggcaggt accagtcccg | 9540 |
| tcgtggtctt gttgctgtta agaggggcat tgcaggcaat gaagttgatc ttcctgatgg | 9600 |
| agactaccaa gtggtgcctc tcttaccaac ttgcaaagat ataaacatgg taaaggtagc | 9660 |
| ttgcaatgta ctgctgagta agttcatagt aggaccacca ggttccggaa aaaccacttg | 9720 |
| gttactgagt caagtccagg acgacgatgt catttacaca cccacccatc agaccatgtt | 9780 |
| tgatatagtc agtgctctca aggtttgcag gtattctatt ccaggggcct ctgggctccc | 9840 |
| ctttccacca cctgccaggt ctgggccgtg ggtcaggctt gttgccagcg ggcacacccc | 9900 |
| cggccgagtg tcatacctcg atgaggccgg gtactgcaac catctggaca ttcttaggtt | 9960 |
| gctttccaaa acaccccttg tgtgtctggg tgaccttcag caacttcacc ccgtcggctt | 10020 |
| taattcctac tgctatgtgt ttgatcagat gcctcaaaag cagctgacca ccatttacag | 10080 |
| gtttggcccc aacatctgtg cagccatcca gccttgttac agggaaaaac ttgaatccaa | 10140 |
| ggccaggaac accaggatag tttttactac acggcctgta gctttcgggc aggtcctgac | 10200 |
| accataccac aaagatcgca tcggttcagc gataaccata gattcgtctc agggggccac | 10260 |
| ttttgacatt gtgactttgc atttaccatc gccaaagtcc ctgaataaat cccgggcact | 10320 |
| tgtggccatc actcgggcaa ggcacgggtt gttcatctac gaccctcaca atcagcttca | 10380 |
| ggagtttttc aacctagctc ctgagcgtac tgattgtaac cttgtgttta accgtgggga | 10440 |
| tgagctagta gtcctgaact cggacaatgc agtcacaacc gtggcgaaag ccctagaggc | 10500 |
| aggcccatct cggtttcgag tatctgatcc gaggtgcaag tctctcttgg ccgcttgctc | 10560 |
| ggccagccta gaagggagct gcatgccgct gccgcaagtg gcgcacaatc tggggttcta | 10620 |
| cttctcccca gatagcccag catttgcacc cctgccgaaa gaactagcgc cacattggcc | 10680 |
| ggtggtcact catcagaaca accgggcatg gcctgaccga cttgttgcta gcatgcgtcc | 10740 |
| aatcgatgcc cgttacagca agccaatggt cggcgctggg tatgtggtcg ggccatccac | 10800 |
| ttttctcggc acccccggcg tggtgtcata ttatctgacg ctgtacgtca ggggtgagcc | 10860 |
| ccaggccttg ccagaaacac tcgtgtcaac ggggcgcata gccacagact gtcgagaata | 10920 |
| tctcgacgcc gctgaggaag aggtagcaaa agaactaccc cacgcattca ttggtgatgt | 10980 |
| caagggtacc acggttgggg ggtgtcatca catcacatca aaacacctac ctaggttcct | 11040 |
| acctaaggat tctgttgccg tggttggagt aagttcaccc ggcaaggctg ctaaagccgt | 11100 |
| gtgcaccctt actgatgtgt acctaccgga actccggcca tatttgcaac ctgagacagc | 11160 |
| gtcaaagtgc tggaagctca aactggactt cagggatgtc cgtctgatgg tctggaaagg | 11220 |
| ggcaaccgcc tattttcaat tagaagggct cacatggtcg gcgctgcccg actatgccag | 11280 |
| gtttattcag ctgcctaagg aagccgtggt gtacatcgat ccgtgtatag gaccggcaac | 11340 |
| agccaaccgc aaagtcgtgc gaaccacaga ctggcgggct gacctggcag tgacaccgta | 11400 |
| tgattacggt gctcagtata ttttgacaac agcctggttc gaagacctcg ggccacagtg | 11460 |
| gaaaattctg gggttgcaac cttttaggcg atcgcttggc tttgagaaca ccgaagattg | 11520 |
| ggcaatcctt gcacgccgta tggatgacgg caaagattac atcgactaca actggaattg | 11580 |
| tgttcaaaac cgtccacttg ctatccacgg gcgcgctcgt gaccacacat atcacttcgc | 11640 |
| cctcggcaca gagttgcagg tggaactggg caaaccccgg ctgccgccgg agcaagtgca | 11700 |
| gtgaacccgg agtgatgcaa tggggtcact gtggagtaaa atcagccagt tgttcgtgga | 11760 |
| cgctttcact gagttcctcg ttagtgtggt tgacattgtc attttccttg ccatactgtt | 11820 |
| tgggttcaca gtcgccgggt ggctactggt cttccttttc agaatggttt gctccgcgat | 11880 |
| tctccgttcg cgctctgcca tttactctcc cgaactatcg aaggtcctat gaaggcctgc | 11940 |
| tacccaactg caggccagat gtcccacaat tcgcattcaa gcatccactg ggcttgcttt | 12000 |
| ggcatatgcg agtttcccat ttgattgatg aaatggtctc ccgtcgtatt taccagacta | 12060 |
| tggaacattc aggtcaagcc gcttggaagc aagtggtcgc tgaggctacc ctcacaaaac | 12120 |
| tatcaaggct tgacgtagtc actcatttcc aacatctggc cgcggtggag gcggattctt | 12180 |
| gccgcttcct cagctcacga cttgcaatgt tgaaaaacct tgccgttggc aatgtgagct | 12240 |
| tgcagtataa caccacattg gacagagttg agctcgtctt ccctacgccg ggttcgagac | 12300 |
| ccaagttgac tgatttcaga caatggctca tcagtgttca cgcttccatc ttctcttctg | 12360 |
| tagcttcgtc tgtcaccttg ttcatagtgc tctggcttcg aattccagcc ttacgctatg | 12420 |
| tttttggttt ccattggccc acggtaatac atcattcgag ctaaccatca actatacaat | 12480 |
| atgtatgccc tgtcttaccc gccaagcggc tagtcagagg ctcgagcctg gccgcaacat | 12540 |
| gtggtgtaga atagggtacg acagctgtga agaacgtgac catgatgagc tgtcaatgtc | 12600 |
| catcccgtct gggtacgaca acctcaaact cgagggttat tacgcttggc tggcattctt | 12660 |
| gtccttctcc tacgccgctc aattccatcc agaattgttc ggaataggga atgtgtcacg | 12720 |
| cgttttcgtg gacaaacaac accaagccat ttgtgctgtg catgacggac aaaattccac | 12780 |
| catatccgct gagtacaaca tttctgcatt gtacgcggca tactaccacc accaagtaga | 12840 |
| cgggggcaac tggtttcatc tagaatggct gcggccattc ttttcttcct ggctggtgct | 12900 |
| caatatttca tggtttctga ggcgttcgcc tgcaagccct gcttctcggc gcatctatca | 12960 |
| gatgttaaga ccaacacaac tgcggctgcc ggtttcatgg tccttcagaa cattgaatgc | 13020 |
| cttcgacacg gagcctcaac aacgcaaaat ggcccttccc ttcggaagcc gtcgcaatgt | 13080 |
| cgtgaagccg ttggcacccc tcagtacatc acaattacgg cgaatgtgac cgatgaatca | 13140 |
| tatttgtaca acgctgactt gctgatgctt tctgcgtgcc tcttttacgc ctctgaaatg | 13200 |
| agcgagaaag gcttcaaagt catcttcggg aacgtttctg gcgttgtctc tgcctgcgtt | 13260 |
| aattttacag actatgtggc ccatgtgact caacacaccc agcagcatca cctggtaatt | 13320 |
| gatcacattc gattgcttca cttcttgtca ccgtctgcaa tgaggtgggc tacaaccatt | 13380 |
| gcttgcttgc tcgccattct cttggcgata tgagatgttc tcacaaactg gagccttcct | 13440 |
| cgactccgca ctcttgctcc tggtggcttt ttttgctgtg taccggcttg ttctggtcct | 13500 |
| ttgccgatgg caacggcaac agcccgacat accaatacat atataacttg acgatatgcg | 13560 |
| agctgaatgg gaccgagtgg ttgtctaacc attttaattg ggccgtcgaa acctttgtgc | 13620 |
| tctacccagt cgcaactcac attatttcac tgggttttct tacaacaagt catttccttg | 13680 |
| atgcgctcgg tctcggcgct gtgtccgtca ccggatttta caacaaccgg tatgtgctaa | 13740 |
| gcagtgtcta ctgcgcctgt gcttttgcag cactcgtgtg ctttgtcatc cgtgccgcta | 13800 |
| aaaattgcat ggcttgccgc tatgcccgca cccggtttac taatttcatc gtggacaacc | 13860 |
| gggggaggat ccaccgatgg aagtctccaa tagtggtgga gaagttgggg aaagctgagg | 13920 |
| ttggtagcga ccttgtcacc atcaaacatg ttgtccttga aggggttaaa gctcaacctt | 13980 |
| tgacgaggac ttcggctgag caatgggaag cttagacaat ttttgcggtg atcctgccgc | 14040 |
| cgtacaaaag cttgtgctgg cctttagcat tacatataca cctataatga tatacgccct | 14100 |
| taaggtgtca cgcggccgac tcttaggact attgcacatc ctaatattct tgaattgttc | 14160 |
| tttcacattt gggtatatga cttatgcgca ttttcaatcc accagtcgtg tcgcgcttgc | 14220 |
| tctgggggct gttgtcaccc tcctgtgggg catttacagt cttacagagt catggaagtt | 14280 |
| tgtcgcttcc agatgcagaa tgtgttgtct aggccggcga tacatcctgg cccctgccca | 14340 |
| tcacgtagaa agtaccgcag gtctccattc aatcccagcg tttggcaacc gagcatacgc | 14400 |
| tgtgagaaag cccggactaa catcagtgaa cggcactctg gtaccaggac ttcggagcct | 14460 |
| cgtgctgggc ggcaaacgag ctgttaaacg aggagtggtt aacctcgtca agtatggccg | 14520 |
| gtaaaagcca gagccagaag aaaaagaaaa atacagctcc aatggggaat ggccagccag | 14580 |
| tcaatcaact gtgccaattg ctgggttcga tgataaggtc ccagcgccag caacctagga | 14640 |
| gaggacaggc gaaaaaaaga aagcctgata agccacattt tcccctagct gctgaagatg | 14700 |
| acattcggca ccacctcacc cagactgaac gttccctctg cttgcaatcg atccaaacgg | 14760 |
| cttttaacca aggcgcaggt gttgcgtcgc tttcatccag cgggaaggtc agttttcagg | 14820 |
| ttgagttcat gttgccggtt gctcatacag tgcgcctaat tcgcgtgact tctgcatccg | 14880 |
| ctagtcagaa tgtagattaa tttgacagtc aggtgaatga ccacgattga cgtgtggcct | 14940 |
| ctaagtcacc tattcaatta gggcgatcac atgggggtca aacttaattg ggcgagaacc | 15000 |
| atgtgaccga aatt | 15014 |

The cDNA consensus sequence of PRRS strain IT 14-32 at P85 has been assigned GenBank Accession number MK024326 (SEQ. ID. NO: 3). The cDNA consensus sequence designated SEQ. ID. NO: 3 is:

| | |
|---|---|
| atgatgtgta gggtatcccc cttgtttttg cagcactcct agtgtttgtg tgcctcggag | 60 |
| gcgtgggtac agccccgccc cacctcttgg cccctgtcct aacccgacag gtacccttct | 120 |
| ccctcggggc gagcgcgccg cctgctgctt tcttgcggcg ggaaggacct cccgagtatt | 180 |
| tctggagagc acctgcttta cgggatctcc accctttaac catgtctggg atgttctccc | 240 |
| ggtgcatgtg caccccggct gctcgggtgt tttggagtgc cggtcaagtc tattgcacac | 300 |
| ggtgtctcag tgcacggcct cttctccctc cagggctgca agacactgac ctcgcagcaa | 360 |
| ttggcttgtt ctacaagccc aaagataaac ttcactggaa agttcctatc ggcatccctc | 420 |
| aggtggagtg tactccgtct ggatgctgct ggctctcggc catcttcccc ttggcgcgca | 480 |
| tgacctccgg caaccacaac ttttctcagc gactcataaa agttgccgaa gtgttgtacc | 540 |
| gtgatggctg tttgactccc cgacaccttc gtgagcttca agtttacgag cgtggttgca | 600 |
| gttggtaccc gatcaccggg cccgtgcccg gagtaggtgt gtacgcgaac tccatgcacg | 660 |
| tgtccgatca atcgtttcct ggtgccactc atgtgctgac gaacttgcct ctacctcagc | 720 |
| aagcttgtcg acagcctttc tgtccattcg atgaggcccg ctctgacgtg tacaagtgga | 780 |
| acgaatttgt ggttttcgtg gattcctcct ccggcggtca attacgcatg atgtggatgc | 840 |
| cgggatccga tgattcggtc gccattgaag cattatcgcc tgagttggaa cgtcaggttg | 900 |
| aaatccttgt tcggagtttc cctgcccacc accctgttaa cattgccgac tgggagcttg | 960 |
| ctgagtcccc ggagcacggt ttttccttcg gcacgtctca tcctagtggt taccttaccc | 1020 |
| gagacccttg gggttttgat ggcaaatgtt ggctctcttg cttcttgggc ctcccgacta | 1080 |
| gagttcagca tcatgaggag tacctagccg acgccttcgg ttaccaaacc aagtggggcg | 1140 |
| tgcacggtag gtatcttcag cgcaggcttc aagtcaacgg tgtccgtgct gtggttgatc | 1200 |
| ctgacggccc catccacgtt gaagcgctgt cttaccccca gtcttggatc aggcacctga | 1260 |
| cttttgacga tgatgttacc ccaggattcg ttcgcctgat gtctcttcgc attgtgccga | 1320 |
| atacagaacc taccactctc ccaattttcc ggtttggggc gcataaatgg tatggagcag | 1380 |
| ctggcaaacg agctcgcgcc aagcgtgctg ccaaaaacaa gggggattcg aattccaccc | 1440 |
| ccgaagtcgc ccgagtggct tctaccagtg aggttgttac ctattcccca ccggcagacg | 1500 |
| ggtcttgtgg ctggcatgtt gttgccgcca tgatgaacca catgatgaac ggtaaactca | 1560 |
| cgtccccttt gactccgtac aacagaccag aggacgactg ggcttctgat tatgatcttg | 1620 |
| tcaagataat tcaatatttg caactgcccg caaccgtagt tcgggcccgt acttgtccca | 1680 |
| acgctaagta ccttgtcaaa ctcaatgggg tccattggga agttgagatg aggccagaag | 1740 |
| tagctccttg ctctctttcc cgcgaatgtg tggttggtgt ctgttctgag ggctgtgttg | 1800 |
| cgtcgcctct tccagaaggg gggctgcctg accgcgcact tgaggccctg gcgtccgctt | 1860 |
| acagattgcc ttccgactgc gttggtgatg ctgttgctga cttcctctcc agcccgccct | 1920 |
| ctcaagaatc ctggaccctc gataaaatgt tgacctcccc atcaccagag cagtccggtt | 1980 |
| tttctagctt gtacaaactg ctactggagg ttgttccgca gaagtgcgga gccacggagg | 2040 |
| gggccttcgt ctatgctgtt gagaggatgc tgaaagattg tccgagcccc aaacaggcca | 2100 |
| tggccctttt gggaaaaatc aaaatcccat cctcaaaagc ctcgtccgtg tccttagacg | 2160 |
| agtgctttcc cactgatgtt ttgcaagagg gtaaccacag gacctcccac cccgtggctc | 2220 |
| ctgctgatgg acttgacaaa cagcaagcac cgttggttga ggatgaacaa ttaggattcg | 2280 |
| gtggtcacga ttcggccgtt gcggcggtca gtggcaatca ggagagtgaa ccgttggacc | 2340 |
| tttcccgatc ggcaccagtt gtaacaacga ccttcgtcga agggcgagtg cccggcgacc | 2400 |
| cgggcccctg caccagcgac cgctccgctg ttgttcaaga gttagttgag cgatgtgacg | 2460 |
| cggagtcaaa tgacggcagt ttgcccctgg atgtgactaa agtgcaaacc cccaatcaac | 2520 |
| ctctggatct atctctagct gcttggccag tgaagaccac tgcatctgac cccggttggg | 2580 |
| ttgacggtag acgcgaaccc gtcttcgtca agcctcgcgg tgctttctct gacagtgagt | 2640 |
| cggtcttccg gtttggagga gtttctgaga ccggccctgt catcgggttt gatcgggtaa | 2700 |
| aagaaattcc ggcggctgac acccccatcg acttaacaat ctcaaaagag actctttccg | 2760 |
| gggcagaccc ctctgagttc gccgcactta agcgcccgcg tttctccgct caagccttga | 2820 |
| ttgaccgagg tggcccactt gctgatgcct gtgcaaagat aaagaatcga gtgtatgagc | 2880 |
| ggtgcctcca ggcttgcgaa cctggcagtc gtgcgactcc agccacaaag gagtggctcg | 2940 |
| acaagatgtg ggaaagggtc gacatgaaga cttggcattg tacctcgcag ttccaaacag | 3000 |
| tctatattct cgggcccctt aaattcctat cagatatgat tagtgacacg ccacctcctg | 3060 |
| tccctaggag ggatcggttt agtgacagtg ccagcttgaa acaattagcg gcacagtggg | 3120 |
| atgagaaatt gaacacagtc cccccccaag ggccggttga gccggggctt agtcgagccg | 3180 |
| ccccctcgcc tgcgaatgcc cagcgagaag gcatcaaccc ctccgatgag ccaccccaaa | 3240 |
| cgccgaaccc ctctggacaa actgttgctg atgggggtgt caaaagactt gtgtccttcg | 3300 |
| gcgtccgcct tgtagggtcc accagccagc gccttatgac atgggttttt gaaatttact | 3360 |
| cccatctccc agcttttatg ctcacactat tctcgccgcg gggctctatg gttgcaggtg | 3420 |
| attggttgtt tgcaggtgtt gtgttacttg ctctcttgtt ttgtcgttct tacccagtgc | 3480 |
| tcggatgcct tcccttattg ggtgtctttt ctggttctgt acggtgtgtt cgtctgggcg | 3540 |
| tttttggttc ttggatggct tttgctgtat ttttattctc aactccaacc aacccagtcg | 3600 |
| gttcttcttg tgaccacgat tcgccggagt gtcatgctga gcttttggct cttgagcagc | 3660 |
| gccaactttg ggaacctgtg cgcggccttg tggtggggcc ctcgggcctc ctatgcgtca | 3720 |
| ttcttggcaa gctactcggt gggtcacgtt atctctggca tgttctctta cgtttatgca | 3780 |
| tgcttgcgga tttggccttt tctcttattt atgtggtgtc ccaagggcgt tgtcacaagt | 3840 |
| gttggggaaa atgtataagg acggctccag ccgaggtggc cctcaacata ttccctttct | 3900 |
| cacgtgccac ccgtgcttcc cttgtatcct tgtgcgatcg gttccaagcg ccaaaagggg | 3960 |
| ttgaccccgt acacttggca acagggtggc gcgggtgttg gcgcggtgag agccctattc | 4020 |
| atcaggcgca ccagaaacct atagcttatg ccaacttgga tgagaagaaa atatccgccc | 4080 |
| aaacagtggt tgctgtccct tatgacccca gtcaggccgt caaatgtttg aaagttctgc | 4140 |
| aggcgggggg ggccatcgta gatcagcccg tacctgaagt ggtccgtgtg tccgagatcc | 4200 |
| ctttttcggc tccattcttc ccaaaagttc cagtcaaccc agattgcagg gttgtggtgg | 4260 |
| attcggacac tttcgtggct gcagtccgct gtggttactc gacaacgcaa ctggtcttgg | 4320 |
| gtcaaggcaa ctttgccaag ttgaacaaca cccctctcaa gaattccgtt tccaccaaga | 4380 |
| cgattggagg ggcctcttac acccttgccg tggttcaggt gtccgtgtgg actcttgttc | 4440 |
| attttgtaat cggtctttgg ttaatgtcgc ctcaagtgtg tggccgaggg acctctgacc | 4500 |
| cttggtgctc aaatcctttt tcatatccta cttatggtcc cggggttgtg tgttcctccc | 4560 |
| ggctttgtgt gtctgccgat ggagtcactc taccattgtt ttcagctgtg gcccaactgt | 4620 |
| ctggtagaga ggttgggatc tttattttgg tgtttgtctc tttaatcgcc ttggctaacc | 4680 |
| gcctagctct taagtctgac atattagtgg tatttctggc actttgtgct tatgcttggc | 4740 |
| ccatgagctc ctggctaatc tgtttctttc ctatactctt gaggtggatc accctccacc | 4800 |
| ctcttaccat gctttgggtg cattcgttct tagtattctg tttgccagcc gccggtgtcc | 4860 |
| tgtcaatagg ggttactggc tttctttggg cgattggtcg tttcacacaa gtcgccggaa | 4920 |
| ttatcacacc ttatgacatt catcaataca cctccgggcc acgcggtgcg gctgctgtag | 4980 |
| caacggcccc agagggtact tacatggcag ccgtccggag ggccgccctg accggacgga | 5040 |
| ctttaatttt taccccatct gcagttgggt ctctccttga gggcgctttc aggacccaca | 5100 |
| agccctgtct caacaccgta aatgtcgtag gttcttccct cggttctgga ggggtcttca | 5160 |
| ctatcgacgg cagaaagact gtcattactg ctgcccatgt attgaacggt gacaccgcta | 5220 |
| gggtcaccgg cgactcctat aatcgcatgc acacttttaa aaccaatggt gattatgcct | 5280 |
| ggtcccatgc tgacgattgg cagggccctt ctcccatagt caaagtcgcg aagaggtatc | 5340 |
| gcggccgcgc ttactggcaa acatcgactg gtgtcgaacc aggcatcatc ggggaagggt | 5400 |
| tcgctttctg tttcaccaat tgtggcgatt caggttcacc tgtcatctct gaagctggcg | 5460 |
| acctcatcgg gatccatact ggttcaaata aacttggatc tggacttgtg accgcccctg | 5520 |
| acggggaaac ctgctccatc aaagagacca aactttctga cctatctagg tactttgcag | 5580 |
| gtccaagcgt ccctctcgga gacataaagt tgagtccagc tatcatccct gatgtggcct | 5640 |
| ctgtcccgag tgacttggca tcacttcttg cttccgtgcc tgtgatggag ggcggccttt | 5700 |
| cgaccgttca acttctgtgt gttttcttcc ttctttggcg catgatgggc catgcctgga | 5760 |
| cccccgtcgt tgctgtgggc ttctttttgc tgaacgaaat cctcccagca gtcttagtcc | 5820 |
| gcgctgtgtt ttcttttgca ctctttgtgc ttgcatgggt caccccctgg tctgcacagg | 5880 |
| tgctgatgat cagactcctc acggcagcac tcaatcgtaa caggttttcc ttggtctttt | 5940 |
| acgcactcgg gggcgtcgtc ggcttggctg ctgagattgg gacttttgct ggtaaactga | 6000 |
| ctgaactgtc ccaggccttg tccacatact gcttcttacc tagggttgct gccatgacta | 6060 |
| gttgcgttcc catcatcatc atcggtgggc tccacaccct tggtgtgatt ctgtggctgt | 6120 |
| tcaaataccg tggtctccac aacatgctgg tcggtgatgg gagtttctca agcgccttct | 6180 |
| ttctgcggta ttttgcagaa ggcaatctaa ggaaaggcgt ttcgcagtct tgtggcatga | 6240 |
| gtaatgaatc tttaacggct gccctggcct gtaagttgtc acaggctgac ctagattttc | 6300 |
| tgtccagcct gacgaacttc aagtgctttg tgtctgcctc aaacatgaaa aatgctgctg | 6360 |
| gccagtacat tgaagcagcg tacgccaagg ccctgcgtca ggagttagct tctctagtcc | 6420 |
| aagttgataa aatgaaagga gtcctgtcca agctcgaagc ttttgctgaa acagcaaccc | 6480 |
| catccctgga cacaggagat gtgattgttc tactcgggca acatcctcac gggtccgttc | 6540 |
| ttgacatcaa tgttgggact gaaaggaaga ccgtgtcagt gcaagagacc cggagcctag | 6600 |
| gcgggtctaa attcagtgtc tgcaccgttg tgtccaacac accagtcgac accttgaccg | 6660 |
| gcatcccact tcaaacgccg accccactct ttgaaaatgg cccgcgccac cgcggtgagg | 6720 |
| atgacgacct taaagttgag aggatgaaga aacactgcgt ctccctcggt ttccataaca | 6780 |
| tcaatggcaa aatttactgc aaggtctggg acaagtccac cggagacacc ttctacacgg | 6840 |
| atgattcccg gtatacccaa gactatgcct ttcaggacag atcggctgat tacagagaca | 6900 |
| gagattatga gggtgtgcaa actgcccctc aacaaggatt tgacccgatg tctgaaaccc | 6960 |
| ccgttggtat tatcgtgatt ggcggtgtca cgtacaacag gtacttagct aaaggcaagg | 7020 |
| aggttttgat ccccaaacct gataaccatc tcgaggccgc taggctttcc ctcgagcaag | 7080 |
| ccctcgctgg gatgggccaa acttgtgatc ttacggctgt cgaggtggag aagttgaagc | 7140 |
| gcattatcag tcaactccaa ggcttgacca ctgagcaggc tttaaactgt tagccgccag | 7200 |
| cggcttgacc cgctgtggcc gcggcggctt agttgtgaca gaaacggcgg tgaagattgt | 7260 |
| gaaataccac agtagaactt tcaccttggg tcctctagac ttgaaagtca cttctgaggc | 7320 |
| ggaagtaaag aaatcaactg agcagggcca cgctgttgtg gcaaacttat gctccggtgt | 7380 |
| cgtcttaatg aggcctcacc cgccatctct cgttgatgta cttctggtgc ccggacttga | 7440 |
| cacagcaccc ggcattcaac cagggcatgg ggccggaaac atgggtgtga acggtgctat | 7500 |
| ttgggatttc gagactgcac ccactaaggc agagctcgag ttgtccaagc agataatcca | 7560 |
| ggcctgtgag gttaggcgcg gggacgcccc gaatctccag ctcctctaca agctttaccc | 7620 |
| tgttaggggg gatcctgaac ggcgcaatgg ctgtctcatc aacaccaggt tcggagattt | 7680 |
| gccctataag actcctcaag acaccaagtc cgcgatccac gcggcttgct gcctgcaccc | 7740 |
| cgatggggcc ccggtgtctg atggcaagtc tacattaggt tccaccctcc aacgtggttt | 7800 |
| tgagctttac gtccccacag tgccttacag tgttttggag taccttgatt cacgccctga | 7860 |
| cacccccctc atgtgtacca aacatggcac ttctgaggct gctgcggagg acctccaaaa | 7920 |
| atacaacctg tccactcaag gatttgtcct gcctggagtc cttcgtttag ttcgcagatt | 7980 |
| cattttcggc catatcggaa aggcgccacc gttgtacctc ccatcaacct atcctgccaa | 8040 |
| aaactccatg gcagggatta atggccaaag gtttccgaca aaggatgtcc agagcatacc | 8100 |
| tgaaattgac gaaatgtgcg ctcgcgccgt caaagagaat tggcagactg tgacgccttg | 8160 |
| caccctcaag aagcagtact gttccaaacc caaaactaga accatcctgg gcactaacaa | 8220 |
| cttcattgcc ttggcgcaca gatcagcact cagtggtgtc acccatgcgt tcatgaagaa | 8280 |
| agcctggaaa tctccaattg ccttgggaaa gaacaaattt aaggagttgc actgtactgt | 8340 |
| cgccggcagg tgccttgagg ccgacttggc ttcctgtgat cgcagcaccc cagccatcgt | 8400 |
| aaggtggttt actgctaatc ttttgtatga acttgcaggg tgtgaggagt acttgcctag | 8460 |
| ctatgtgctc aactgctgtc atgatctcgt ggcaactcag gatggcgctt ttacaaagcg | 8520 |
| tggtggtctg tcgtctgggg accccgttac cagtgtgtcc aacactgtgt actcactggt | 8580 |
| gatttacgcc cagcacatgg tgctgtcagc actgaagatg ggccacgaaa ttggcctcaa | 8640 |
| gttcctcgag gaacaactca agttcgagga ccttattgaa attcagccca tgttggtgta | 8700 |
| ctctgatgac ctcgtcttgt atgctgagaa gcccaccttt cctaattacc actggtgggt | 8760 |
| cgagcacctt gatttgatgc tgggtttcaa gacggaccca aaaaaaacta ttataactga | 8820 |
| caaacccagc tttctcggct gcagaattga ggcagggcgg cagttagtcc ccaatcgcga | 8880 |
| ccgcatcctg gccgcccttg cataccacat gaaagcgcag aacgcttcag aatattacgc | 8940 |
| gtctgctgcc gcaatcctga tggattcatg tgcttgtatt gactatgacc ctgagtggta | 9000 |
| tgaggatctc atctgcggca ttgcccggtg cgctcgtcaa gatggctata gttttccagg | 9060 |
| cccgccattt tttatgtcca tgtgggaaaa gctgaaaagt cacaatgaag ggaaaaaatt | 9120 |
| ccgccactgc ggtatctgtg atgccaaggc tgaccatgcg tccgcctgcg ggcttgattt | 9180 |
| gtgtttgttc cattctcact ttcatcagca ttgcccagtc atgcttagct gtggtcatca | 9240 |
| cgctggttta aaagaatgcc cgcagtgtca gtcaccagtc ggggctggca agtcccctct | 9300 |
| tgacaccgtg ttgcaacaaa tcccgtataa accaccccga actgtcataa tgaaggtgaa | 9360 |
| cagtaaaaca acagcccttg acccggggag gtatcagtcc cgtcggggtc ttgtcgcagt | 9420 |
| caagagggga attgcaggca atgaggttga tctcgctgac ggggactacc aggtggtacc | 9480 |
| cctcctgccg acctgcagag acataaatat ggtgaaggtg gcttgcaatg tactactcag | 9540 |
| caaattcata gtagggccac ccggctccgg aaagactacc tggttgctga accaagtcca | 9600 |
| agatgatgat gttatctata cacccaccca tcagaccatg tttgatatag tcagtgctct | 9660 |
| taaggtttgc aggtattcaa tcccaggagc ctcaggactc ccttttccac cgcctgccag | 9720 |
| atccggacca tgggtcaggc ttatcgccag cgggcacatc cctggccggg tctcatacct | 9780 |
| tgacgaggcc gggtattgca atcatctgga catcctcaga ctgctttcca aaacacccct | 9840 |
| cgtgtgtttg ggcgatcttc aacaacttca ccctgtcggc tttgattcct gttgttatgt | 9900 |
| ttttaatcag atgccacaca aacagctgac caccatttac aggttcggcc ctaatatctg | 9960 |
| tgccgccatc cagccttgtt acagggagaa gcttgaatcc aaggcaagaa acaccagggt | 10020 |
| ggttttcacc actcaacctg tggcctatgg tcaagtgctg acaccatttc acaaggatcg | 10080 |
| cgtagactca gccataacca tagattcatc tcagggtgcc acctttgacg tcgtgacgtt | 10140 |
| acacttgccg acgccaaaat ccctgaacaa atcccgagca cttgtggcta tcacccgggc | 10200 |
| gaggcatggg ctgttcatct atgatcctca taaccaactt caggagttct tcaacctaac | 10260 |
| ccctgagcgc acggattgca accttgtgtt caaccgtggg gatgaactgg tcgtcctgga | 10320 |
| tccagataat gcagtcacga ccgtagccaa ggccctgggg gccggcccgt ctcaattccg | 10380 |
| ggtgtccgat ccgaggtgca agtccctctt ggccgcttgc tcagtcagcc tggaaggtgg | 10440 |
| ctgcatgccg ctgccgcagg tggcccataa tttggggttt tatttctccc cagacagtcc | 10500 |
| ggcgtttgca cctctgccaa aagaactggc accacattgg ccggtggtta cctgccagaa | 10560 |
| caaccgggca tggcctgatc gactcgttgc cagcatgcgc ccgattgacg cccgttacag | 10620 |
| caagcctatg gttggtgcgg ggtatgttgt tgggccgtcc acctttcttg gaacccctgg | 10680 |
| tgtagtgtca tattacctca cactgtacat caagggtgag ccccaagccc taccagaaac | 10740 |
| actcgtttcg acagggcgta tagccacaga ctgccgggaa taccttgata cagctgaaga | 10800 |
| agaagcagct aaagaactcc ctcatgcttt cataggtgat gtcaaaggca ccacagtagg | 10860 |
| ggggtgtcac cacattacat caaaatactt acccaggtcc ctacccaagg actctgttgc | 10920 |
| agtggttggg gtgagctcac ctggcaaggc tgccaaagcg gtgtgcactc tcactgatgt | 10980 |
| gtacctccct gaccttcggc cgtatctgca accagagaca gcatcaaaat gctggaaact | 11040 |
| caaattggac ttcagggacg tcagattgat ggtttggaaa ggggccactg cctatttcca | 11100 |
| attggaagga ctcacatggt cggcattacc cgactatgcc aggttcattc agctacccaa | 11160 |
| agatgccgta gtatacattg acccgtgtat aggaccggca acggccaacc gcaaggttgt | 11220 |
| gcgaaccact gactggcgag ctgacctggc ggtaacaccg tatgattacg gtgcccagac | 11280 |
| tattctgaca acagcctggt tcgaggacct cggaccacaa tggaaaatcc tggggctgca | 11340 |
| gccctttaag cgggcatttg gcctcgaaaa cactgaagac tgggcaatcc tcgcacgccg | 11400 |
| tatgagtgac ggcagagatt acaccgacta caactggact tgtgttcgag aacgcccgca | 11460 |
| cgctatctac gggcgcgctc gtgaccacac gtatcatttc gcccctggca cagaattgca | 11520 |
| ggtagaactg ggcaaacccc ggctgccgct tgagtgagta ctgcggctcg aaagccatgc | 11580 |
| aatggggtca ctgtggagta agatcactca gctgtttgtg gatgccttca ctgaattcct | 11640 |
| tgttagtgtg gttgacattg ttatcttcct tgccatacta ttcgggttca cagtcgcagg | 11700 |
| atggttactg gtctttctac tcagagtggt ttgctccgcg attctccgtt cgcgcactgc | 11760 |
| cattcactct cccgaattat cgaaggtcct atgaaggcct gctgcccaat tgcagaccgg | 11820 |
| atgttccaca attcgcaatc aagcatccat tgggcatcct ttggcacatg cgagtttccc | 11880 |
| atctaattga tgaaatggtc tctcgtcgtg tctaccaaac catggaacat tcaggccaag | 11940 |
| cggcctggaa gcaggcagtt gctgaagcca cacttacaaa gctatcgcag cttgacatgg | 12000 |
| tcacccactt ccagcatctt gccgcagtgg aagcggattc ttgtcgcttc ctcagttcgc | 12060 |
| gactcgtgat gctaaaaaac cttgctgtcg gcaatgtaag tctgctgtac aataccacat | 12120 |
| tggaccgtgt tgaactcatt ttccccacgc caggtgcgag gcccaaattg accgatttca | 12180 |
| gacaatggct catcagtgtt catgcttcta ttttctcttc tgtagcttcg tcagtcactt | 12240 |
| tgtttgtagt gctttggctt cgaattccag tgctacgcta tgtttttggt ttccattggc | 12300 |
| ccacggcaac acgtcgttcg aactgaccat caattacacc gtatgcaagc cttgcattac | 12360 |
| tagacaagct gctgcccaac gactcgagcc tggtcgtaac atgtggtgca aaattgggta | 12420 |
| cgatcactgt gaagagcgtg atcacgatga gttgtcaatg gccatcccgt ccgggtacga | 12480 |
| caacactaaa cttgaaggct attatgcttg gcttgccttc ttgtcttttt cttatgcggc | 12540 |
| ccagtttcat ccagagctgt ttggaatagg gaatgtgtca cacgttttcg tggacaagca | 12600 |
| gcatcaattc atctgtgccg agcatgatgg gcaaaattca accataccca acccacacaa | 12660 |
| catctctgca ttgtatgcgg tgtattatca ccaccaagtg gacgggggca actggttcca | 12720 |
| tctagaatgg ctgcggccat tcttctcctc ctggttggtg ctcaatattt catggtttct | 12780 |
| gaggcgttcg cctgcaagcc ctgtttctcg acgcatctat cagatattaa aaccaacaca | 12840 |
| accgcggctg ccggtttcat ggtccttcaa gacattagct gttcccaacc ccatgagaga | 12900 |
| tcgggcacgc ggtcgtccgt tcgcgggaag ccatcccaat gtcgtacagc catcggcacc | 12960 |
| cccctgtaca tcacgctaac ggcgaatgtg actgatgaat cttatttgta caatgctgat | 13020 |
| ttattgatgc tttctgcttg cctgttttac gcctcggaaa tgagtgagaa gggcttcaaa | 13080 |
| gtcatttttg gaaacgtctc tggtgttgtt tccgcgtgcg tcaatttcac agactatgtt | 13140 |
| acccatgtga ctcagcatac ccaacagcat catctggtag tcaaccatat ccggttgctg | 13200 |
| cactttatga caccgtcaac gatgaggtgg gccacaacca tcgcttgttt gctcgccatt | 13260 |
| ttattggcga tatgagatgt tttcccagat tggggcgttt tttgactcct cactcttact | 13320 |
| tctggtggct ttctttgttg tgtaccggtt tatcctggtc ctttgccgat ggcgacggca | 13380 |
| acagcccgac ataccaatac atatataact tgacgatatg tgagctgaat gggaccacct | 13440 |
| ggctgtccaa caatttttac tgggcagtcg aaacttttgt gctttacccg gtggtgaccc | 13500 |
| acatcgtctc actgggtttc ctcacaacta gccatttctt tgacgcgctc ggcctcggag | 13560 |
| ctgtgtccgc tgtgggattt gctggcgggc ggtatgtcct cagcagcata tacggcgttt | 13620 |
| gtgcattcgc agcgctcgtg tgtttcatca tccgtgttgt taaaaattgc atggcgtgcc | 13680 |
| gctatgcccg tactcggttc accaacttca ttgttgacga ccgaggaaga attcacagat | 13740 |
| ggaagtcccc aatagtggtg gaaaaaatgg gtaaggccga agttggcagc agtctcgtca | 13800 |
| ccatcaagca tgttgttctc gaaggggtta aagctcaacc cttgacgagg actccggctg | 13860 |
| agcaatggga agcctagatg acttttgcta tgattccgcc gctacacaaa agcttctact | 13920 |
| agctttcagt atcacataca cacctgttat gatatacgcc cttaaggtat cacgcggccg | 13980 |
| acttttgggg cttttgcaca tcttgatttt cctgaattgt tccttcacat tcggatacat | 14040 |
| gacccatgag cgtttccatt ccaccaatcg tgtggcgctt actatgggag ctgttgtcgc | 14100 |
| tctcctgtgg ggcatctata gcctcacaga atcatggaag tttattactt ccaggtgcag | 14160 |
| attgtgttgc ctaggccggc aatacatcct ggcccctgcc caccacgtag aaagtgccgc | 14220 |
| aggactccat tcaatcccgg cgtctggcaa ccgagcatac gccgtgagaa agcccggatt | 14280 |
| aacatcagtg aacggcactt tagtaccagg gcttcggagc ctcgtgttgg gcggcaagcg | 14340 |
| agctgttaaa cgaggagtgg ttaacctcgt caaatatggc cggtaaaaac cagagccaga | 14400 |
| agaagaagaa aaatacagct ccaatgggga atggccagtc agtcaatcaa ctgtgtcagt | 14460 |
| tgctgggcac gatgatgaga tcccagcgcc agcgatccaa ggggggacag gccaaaaaga | 14520 |
| aaaagcttga gaagccgcat ttccccctgg ccgctgaaga tgatgtccgg caccacctca | 14580 |
| cccaaaccga acgttccctt tgtctgcaat cgatccagac agcctttaat caaggtgcgg | 14640 |
| gaactgcgtc gctttcatcc agtgggaagg tcggttttca ggttgagttt atgctgccgg | 14700 |
| ttcctcatac ggtgcgcctg attcgcgtga cttccacatc cgccagtcag ggtgcaaatt | 14760 |
| aatttgatag tcaggtgaat ggccacgatt ggcgtgtggc ctctgagtca cctattcaat | 14820 |
| tagggcgatc acatgggggc tagacttaat caggcgagaa ccatgtgacc gaaatt | 14876 |

The cDNA consensus sequence of PRRS strain PL 14-02 at P85 has been assigned GenBank Accession number MK024327 (SEQ. ID. NO: 4). The cDNA consensus sequence designated SEQ. ID. NO: 4 is:

| | |
|---|---|
| atgatgtgta ggggagatac cctacacaca caacactcct agtgtttgtg taccttggag | 60 |
| gcgagggtac agccccgccc cactccttgg cccctgtttc agcccaacag ggacccttct | 120 |
| ccctcggggc gagtgtgccg cctgctgctc tctcgcagcg ggaaggacct cccgagtatt | 180 |
| tccggagagc acctgcttca cgggatctcc accctttaac catgtctggg atgttctccc | 240 |
| ggtgcatgtg caccccggct gcccgggtat tttggaacgc cggccaagtc ttttgcacac | 300 |
| ggtgtctcag tgcgcggtct cttctccctc tagagcttca ggactctgac ctcggcgctg | 360 |
| ttggctgttt ttataagcct agggacaagc ttcattggaa ggtccccatg ggcatccctc | 420 |
| aggtggagtg cactccatcc ggatgctgtt ggctcgcagc catttttcct atagcgcgta | 480 |
| tgacctccgg caatcacaat ttctcccagc gacttgtgaa agttgctgat gttttgtacc | 540 |
| gcgacggttg tctaacacct caacaccttc gtgaactcca agtttacgaa cgcggctgca | 600 |
| actggtaccc gatcacgggg ccagtgcccg ggatgggttt atttgcgaac tccgtgcacg | 660 |
| tgtctgacca gccgttcccc ggcgccactc atgtgttaac caactcacca ttgcctcagc | 720 |
| aggcttgtcg acagccgttc tgtccatttg aggtggctca ttctaacgta tacaggtggg | 780 |
| gggagtttgt gatctttgtg gactctcctt ccggcggtcg atcgcgtatg atgtggacac | 840 |
| cgggatccag tgactcggct gccctagaag cgttaccgtc cgcattagaa cgtcaggccg | 900 |
| gagtccttgt tcgaagtttc cctgcccacc accccgtcga ccttgctgac tgggagctta | 960 |
| ccgaatcccc tgaacacggt ttttccttca gcacatttca ttcttgtggt taccttgctc | 1020 |
| aaaaccccga aagttttgac ggtaagtgct ggctttcctg cttcctgggc ttgccgaccg | 1080 |
| gcgtgcggcg ttgtgaggaa tttttagctg gcgcctttgg ttatcaaacc aaatgggggg | 1140 |
| tgcacggaaa gtacctccaa cgtaggcttc agattaacgg ccttcgcgct gtggtcgatc | 1200 |
| ctgacggtcc catccatgtt gaagcacttt cttgtcccca gtcttggatc aggcatctga | 1260 |
| cttttgacga tgatgtcacc ccgggattcg ttcgcttaac atcccttcgc attgtgtcaa | 1320 |
| atacagagac cacctcctcc cagatctttc ggttcggggc gcataagtgg tacggcgctg | 1380 |
| ccggcaagcg ggctcgcgct aagcgtgctg caaagagtga gaagaaatcg gttcccaccc | 1440 |
| ccgagactgt tccgctgacc cccgcctgtg gagtcaccat ctattcccca ccggcagacg | 1500 |
| ggtcctgtgg ttggcatgcc cttgccgcca tattgaacca gatgatgaac ggtgacttca | 1560 |
| cgtccccttt gcctcagtac aacaggccag aggatgattg ggcctctgac aacgaccttg | 1620 |
| ctcaagcaat tcaactcctg cgactaccgg ctaccatagt tcggactcgt gcctgtccta | 1680 |
| atgccaggta ccttataaag ctgaatgggg ttcactggga agtggaggag aggctgggga | 1740 |
| cggctctttg ctccctttct cgtgaatgtg tggttggtgt ttgctctgaa ggctgtgtcg | 1800 |
| catcgcctta tccaacagac ggggtaccag agcgtgcact cgaggccttg gcatctgctt | 1860 |
| acagactacc ctccgattgt gtttgctctg gtatttctga cttccttacc gaaccgcctc | 1920 |
| ctcaggaact ctggactctc gacaggatgt tgacctctcc atcacctgag cggtccggct | 1980 |
| tctccagttt gtataaatta ctgctcgagg ttgtccctca aaaatgcggt gctacggaag | 2040 |
| gggctttcac ctatgctgtt gagagaatgt tgagggactg ccgaagctcc aaacaagcca | 2100 |
| tggctctttt ggcgaaaatt aaagttccgt cttcaaaggc ttcatctgtg tccctggacg | 2160 |
| agtgtttccc tacgggtgct ccaggtgatt ctgagccagc acttcaggag gggcctcgaa | 2220 |
| gcctcggtgc tgccgttgtc ccatgcctgc ctggtgcaaa aggattcgag gaagcagccc | 2280 |
| cggaaggggt tcatgagaat ggctacgatg ccacccaccc tgcgctcttt gctgagcgtt | 2340 |
| ctaccaacga gcaggcacga atggcagccg gtaggcaatt ggggttcagc gatcgtgatt | 2400 |
| tggcagtcaa gaacattaat gaaggtgatt cggtctcggt tggtccaaca gaaggcacac | 2460 |
| tcaatggtca gggagacgaa ccactggatt tgtcccgacc agcactgacc actacaacga | 2520 |
| cccttatggg agaacgagaa cccgacaacc ctggttctga tgccggtgct tcccctgata | 2580 |
| ctgttcgaga attttccttg acggggctca cattccgtca tgttgagcac tgtggtacgg | 2640 |
| agtcgggtga cagcgattcg cctttggatt tgtctgacac gcagacccag gaccaacctt | 2700 |
| tagatctatc cctggcctct tggccagtga aagctaccgc atctgacccc ggctgggttc | 2760 |
| acggtaggcg tgagcctgtc tttgtgaagc ctcgaaatgc tttctctgat ggcgattcag | 2820 |
| tccttcagtt tggagggctt tctgaacccg gctctgtcat cgagcctgac cagataaaag | 2880 |
| gtgccccggt gactgacacc cctaccgacc taacgacttc tgacgagtcc tttcccgcag | 2940 |
| gtgatcctct tgaactcgct gagcttaagc gcccacagtt ctccgtacag gccttaattg | 3000 |
| atcgaggggg cccacttgct gcattttatg caaaaataaa gaatcgggta tatgaacagt | 3060 |
| gcctccaagc ctgtgagccc ggtagtcgtg caaccccggc cactaaggag tggctcaaca | 3120 |
| aaatgtgggg tagggtagac atgaagactt ggcgttgtac ttcgcagttc gaagctggtc | 3180 |
| gctctcttgc gtcccttgaa ttcctccctg atatgatcaa cgacactcca ccccctgttc | 3240 |
| ccaggaataa ccgggtcagt gacgacgccg gcttgaagca actggtagca caatgggata | 3300 |
| agaaattgag cgcacccccc cccccaaaac tggttgggcc agtgatcgac cagagcgccc | 3360 |
| tcccaccagc aggtgtccaa caggaaaata tcaccccttc cgatgggccg cctcaagcgc | 3420 |
| cggattttcc tggtcgagcg ggtacaggca gaggttggaa aggctttgtg ctttccggca | 3480 |
| ctcgtcttgc agagtctgtt agtcagcgcc ttatgacgtg ggtttttgaa gtctactccc | 3540 |
| atctcccagc ttttatgctc gcacttttct cgccacgggg ctctatggtt ccaggtgatt | 3600 |
| ggttgtttgc aggtgttgtg ttacttgctc tcttgctctg tcgttcttac ccagtactcg | 3660 |
| ggtgtctacc cttactgggt gtattttctg gttctctacg gtgtgttcgt ctgggtgttt | 3720 |
| ttggttcttg gatggctttt gctgtatttt tattctcaac tccagccgat ccagtcggtt | 3780 |
| cttcttgtga ccacgattcg ccgaagtgtc atgctgagct tttggctctt gagcagcgcc | 3840 |
| aactttggga acctgtgcgc ggccttgttg tgggcccctc gggtctctta tgcgtcattc | 3900 |
| ttggcaagtt actcggtggg tcacgttatc tctggcatgt tttcttacgt ttatgcatgc | 3960 |
| ttgcggattt ggccctttct cttgtttatg tggtgtccca ggggcgttgt cgcaggtgtt | 4020 |
| ggggaaagtg tataaggaca gctcctatgg aggtggccct caacgtattc cctttttcgc | 4080 |
| gtgccacccg ctcctctctc gtgtccttgt gtgatcgttt tcaaacgcca aaaggggttg | 4140 |
| atcctgtgca cctggcaacg ggttggcgcg ggtgctggtg cggtgagagt cccatccatc | 4200 |
| aatcacatca aaagcccata gcttatgcca atttggatga aaagaaaata tctgcccaaa | 4260 |
| cagtagtcac tgtcccatat gatcccagtc aggctgtcaa gtgcctaaaa gttttgcagg | 4320 |
| cagggggggc catcgtagac cagcccacac ctgaggtcgt tcgtgtgtcc gagatcccct | 4380 |
| tctcagcccc atttttccca aaagttcccg tcaacccaaa ttgccgggtt gtagtggatt | 4440 |
| cagacacttt cgtggccgca gtccgatgcg gttactcaac agcacaactg gttctaggcc | 4500 |
| gtggtaactt tgctaagtta aatcaggccc ctcccaaaaa ctctgccctc accaaagcaa | 4560 |
| ctggtggggc ttcttacacc cttgctgtgg ctcaagtgtc tgtgtggact cttgttcatt | 4620 |
| tcatccttgg catttggctc acaacacctc aagtgtgtgg acgagggacc gtcgacccat | 4680 |
| ggtgtacaaa ccccttttcg taccccactt acggccccgg agttgtgtgc tctgctcgac | 4740 |
| tttgtgtgtc cgccgacggg gttaccctgc cgttgttctc tgccgtggca cagctctccg | 4800 |
| gtagggaggt agggatcttc attttggtac tcgcctcttt gggtgctctt gtccaccgcc | 4860 |
| tggctcttaa ggcagacatg ttaatggtct ttttggcttt ttgtgcttac gcctggccta | 4920 |
| tgagctcctg gttgatttgc ttcttcccca tactcttgaa gtgggtcaca cttcaccccc | 4980 |
| tcaccatgtt ttgggtacac tcatttttag tgttttgtct gccagcagcc ggtatcctct | 5040 |
| cactgggagt gactggtctt ctctgggcaa ttggccgctt tacccaggtt gccggaatta | 5100 |
| ttacacctta tgacatccat cagtacacct ctgggccacg tggtgcggcg gctatagcca | 5160 |
| cagcccctga aggcacttac atggccgccg tccggagagc cgctttaact gggcgaactt | 5220 |
| tgatcttcac cccgtcagca gtaggatccc ttcttgaggg agcctttagg acccgtaaac | 5280 |
| cctgcctcaa caccgtgaat gtcgtgggct cttcccttgg ttcaggaggg gttttcaccg | 5340 |
| ttggtggaaa gaaaataatc gtcaccgcag cccacgtgct gaatggtgac acggctagag | 5400 |
| tcaccggtga ctcctacaac cgcttgcaca ccttcaatac taatggtgat tacgcctggt | 5460 |
| ctcatgctga agactggcag ggcgttgccc ctgcagtcaa cattgcgaag gggtaccgcg | 5520 |
| gccgcgccta ttggcaaacg tcaaccggtg tcgagcccgg agttgttggg ggagggtttg | 5580 |
| ccttctgttt tactaactgc ggagactcgg ggtcacctgt tatctcagaa tctggtgatc | 5640 |
| ttattgggat tcacaccggc tcaaacaaac tcggctctgg tcttgtaaca acccctgaag | 5700 |
| gggaaacctg cactattaaa gaaaccaagc tctctgacct ttctaaacac tttgcaggcc | 5760 |
| caagcgttcc cctcggggac atcaaattga gtccggccat tatccctgac gtgacatcca | 5820 |
| ttccgagtga cttggcgtcg ctcttgactt ctgtccctgt aatggaaggc ggcctctcga | 5880 |
| ccgtccaact tttgtgtgtc tttttccttc tctggcgcat gatgggccat gcctggacac | 5940 |
| ccgttgttgc cgtgggcttc ttcctgctga atgaaatcct cccagcagtc ttggtccgga | 6000 |
| ctgtgttttc ttttgcactc tttgtgctgg catgggcaac cccttggtct gcacaggttt | 6060 |
| taatgatcag gcttctcacg gcatctctca atcgtaacag gtcttcttta gtgttctacg | 6120 |
| catttggagg catcgtcggc ttggccgttg aaatcgggac cttctctggc agattgcctg | 6180 |
| cattgtctca agccctttcg acatattgct ttttacccag agcccttgtc atgaccagct | 6240 |
| gtgtccccac cattatcatt gggggattcc atatcctcgg tgtaatcttg tggttgttca | 6300 |
| aataccggta cctccacaac atgctggtcg gtgatgggag tttttcaagc gcctttttcc | 6360 |
| tacggtattt tgctgaaggc aatctcagga agggtgtctc aaagtcttgt ggcatgagta | 6420 |
| acgagtccct aacggctgca ctggcctgca aattatcaca ggctgacctt gattttctat | 6480 |
| ccagcttgac gaacttcaag tgctttgtat ccgcttcaaa catgaaaaat gctgccggcc | 6540 |
| agtacattga agcagcgtat gccaaggccc tgcgccaaga gttggcctcc ctagttcaag | 6600 |
| tcgacaaaat gaaaggagtc ttgtccaagc tcgaagcttt tgctgagaca gccactccgt | 6660 |
| cccttgacgc gggtgacgtg atcgttcttc taggacaaca tcctcacggg tctgttctcg | 6720 |
| acatcaatgt gggggctgaa aggaaaactg tatctgtaca agagacccgg agtctaggcg | 6780 |
| gctccaaatt cagcgtctgc actgtcgtgt ccaacacacc catcgatgcc ttaactagta | 6840 |
| ttccactcca gacaccaacc ccactcttcg agaatggtcc gcgtcatcgt ggcgaggagg | 6900 |
| acgatctcaa agttgagagg atgaagaaac actgtgtgtc cctcggcttt cacaacatca | 6960 |
| atggcaaagt ttactgtaaa atttgggaca agtctaccgg cgacaccttc tacacagatg | 7020 |
| attcccggta tacccaggac tatgcttttc aagacagatc agtcgattac agggacaggg | 7080 |
| actatgaggg cgtacaaacc gtctcccatc agggattcga tccaaagtcc gaaacccctg | 7140 |
| tcggcactgt tgtgatcggc ggcattacgt ataacaggta tctgacaaag ggtaaagaaa | 7200 |
| ttctggttcc caggcctgac aactgccttg aagctgctaa attgtcctta gagcaagctc | 7260 |
| tcgccgggat gggtcagact tgcgaactta cgaccgccga gatggaaaag ttgaagcgca | 7320 |
| tcattagtca actccaaggt ttgaccactg aacaagcttt aaactgctag ccgccagcgg | 7380 |
| cttgacccgc tgtggccgcg gcggcttagt tgtgactgaa acggcggtaa aaattgtgaa | 7440 |
| ataccatagt agaactttta ccttaggctc cttagacctg aaggtcgctt ccgaggtaga | 7500 |
| ggttaaaaag tcagctgagc aaggccacgc tgttgtggca aacttatgct ccggtgtcat | 7560 |
| tttgatgaga ccccacccac cgtcccttgt tgatgttctt ctgaaacccg gacttgacac | 7620 |
| aaaacccggc attcaaccag ggcatggggc cgggaacatg ggcgtggatg gttctacttg | 7680 |
| ggattttgag accgcgccca caaaggcaga gcttgagtta tcgaagcaga taatccaagc | 7740 |
| atgtgaagtt aggcgcggag acgctcccaa cctccaactc ccttacaaac tttaccctgt | 7800 |
| cagggggaac cctgagcggc atgacggccg tctcactaac accaggtttg gagatttgcc | 7860 |
| ttacaagacc cctcaggaca ctaaatccgc aatccacgcg gcttgttgcc tgcatcccaa | 7920 |
| tggggtcccc gtatctgatg gcaaatctac actaggtacc actcttcaac atggtttcga | 7980 |
| gctttatgtc cctaccgtgc cctatagtgt catggagtac cttgattcgc gttctgacac | 8040 |
| ccctctcatg tgtactagac atggcacttc caaagctgcc gcagaggacc tccaaaagta | 8100 |
| tgatttatcc acccaggggt ttgtcctgcc tggggtccta cgccttgtgc gcaagtttgt | 8160 |
| ttttggccat gttggtaagg caccgccatt gttccttcca tcaacctacc ccgcaaaaaa | 8220 |
| ctccatggca ggggtcaatg gccaaaggtt tcctacaaaa gatgttcaga gcatacctga | 8280 |
| agttgatgaa atgtgtgctc gcgccgtcaa ggagaattgg caaactgtga cgccttgtac | 8340 |
| tcttaagaaa cagtactgct ccaagctcaa aaccagaacc atcctgggca ccaacaattt | 8400 |
| tattgcccta gcccacagat cggcgctgag tggcgtcacc caggctttta tgaagaaggc | 8460 |
| ctggaattcc ccaattgcct tggggaaaaa taaattcaaa gagctgcact gtgttgtcgc | 8520 |
| cggcaggtgc cttgaggctg atctggcctc ctgtgaccgc agcacccctg ccgttgtaag | 8580 |
| atggtttgtt gccaacctcc tgtacgaact cgcggggtgt gaagagtatt tgcctagcta | 8640 |
| tgtgctcaat tgctgccatg atctcgtagc aacacaggat ggtgccttta cgaaacgcgg | 8700 |
| tggcctgtcg tctggggacc ccgtcactag cgtgtccaac actgtgtatt cactgataat | 8760 |
| ttatgcccag catatggtgt tatcagcttt gaaaatgggt cacgaaattg gtctcaagtt | 8820 |
| ccttgaggaa cagcttaaat tcgaagacct tcttgaaatt cagcccatat tggtgtattc | 8880 |
| tgatgacctc gtattgtatg ccgaaagacc aacttttccc aactaccact ggtgggttga | 8940 |
| gcatcttgac ctaatgctgg gcttcaaaac ggacccggca aaaaccgtca taaccgacaa | 9000 |
| gcccagcttc ctcggctgta gaattgaggc agggcggcag ctagttccca atcgcgaccg | 9060 |
| catcctggct gctctcgcat accacatgaa ggcgcagaac gcctcagagt attatgcgtc | 9120 |
| tgctgctgca atcctaatgg attcatgtgc ttgcattgac cacgaccctg agtggtatga | 9180 |
| ggacctcatt tgcggtattg cccggtgcgc ccgccaggat ggttacagct tcccaggtcc | 9240 |
| accatttttc atgtctatgt gggagaaatt gagaagtcat aatgagggga agaaattccg | 9300 |
| ccattgcggc atctgcgacg ccaaagccga ccatgcgtcc gcttgcgggc ttgatttgtg | 9360 |
| tttgttccat tcgcactttc atcaacactg cccagtcact cttaactgcg gtcatcatgc | 9420 |
| cggttcaaag gaatgttcgc ggtgccagtc acctgttggg gctggaaaat cacctcttga | 9480 |
| tgccgtgcta gaacaaattc catacaaacc tcctcgcact gtcatcatga atgtgaacag | 9540 |
| tggaacgacg gcccttgatc cagggaggta ccagtcccgt cgtggcctcg ttgcagttaa | 9600 |
| gaggggtatc gcaggtaatg aagttgatct ccctgatgga gactatcaag tggtgcctct | 9660 |
| tttaccgact tgcaaagaca taaacatggt gaaggtggct tgcaatgtgc tgctcagtaa | 9720 |
| gttcatagtg gggccgccag gttccgggaa aaccacctgg ctattgagtc aagttcagga | 9780 |
| cgatgacgtc atttacacac ctacccacca gaccatgtat gacatagtta gcgccctcaa | 9840 |
| ggtttgtagg tattctatcc caggagcctc gggacttcct tttccgccac ctgccagatc | 9900 |
| cgggccgtgg gttaggcttg ttgctagcgg ctacgtccct ggccgagtgt catacctcga | 9960 |
| tgaggctgga tattgcaatc acctggacat tcttagactg ctttctaaaa caccccttgt | 10020 |
| ttgtttgggt gacctccagc aacttcaccc tgtcggcttt gattcctact gttatgtgtt | 10080 |
| tgatcagatg cctcagaagc agttgactac catttataga ttcggtccta acatctgtgc | 10140 |
| agccatacag ccttgttaca gggagaaact cgaatccaag gctaggaata ccagggtggt | 10200 |
| cttcaccacc cgacctgtgg cctttggcca ggtgctgaca ccataccata aagatcgcgt | 10260 |
| cgactccgcg ataaccatag attcgtcaca gggggccacc ttcgacattg taacgttgca | 10320 |
| tttgccatca ccaaaatcct taaacaaatc ccgagcactt gtagccatca ctcgggctag | 10380 |
| gcacgggttg ttcatttacg atccccataa ccagctcagg gagtttttca acctgacccc | 10440 |
| tgagcgtact gaatgcaacc tcgtgttcag ccgtggggat gagctggtgg ttctgaacgc | 10500 |
| ggacaatgca gtcacgactg tagtaaaagc cctagaggtg ggttcatccc agtttcgagt | 10560 |
| gtcagacccg aggtgtaaat ctctcttagc tgcttgttca gccagtctgg aggggagctg | 10620 |
| tatgccgtta ccccaagttg ctcataacct agggttttat ttttcccctg acagtccggc | 10680 |
| atttgcaccc ctgccaaaag agttggcacc acattggcca gtagttactc accagaataa | 10740 |
| tcgggcatgg ccagaccggc tcgtcgctag catgcgccca attgatgctc gctacagtaa | 10800 |
| accaatgatt ggtgctggtt atgtagtcgg accatccacc tttctcggca ctcctggcgt | 10860 |
| agtgtcatac tatctcaccc tatacattag gggtgagccc caggccttgc cagaaacact | 10920 |
| cgtgtcaaca ggacgcatag ccacagattg tcgggagtat cttgatacag ccgaggaaaa | 10980 |
| tgcggcaaaa gaactccctc acgcatttat tggtgatgtg aaaggcacta cggtcggtgg | 11040 |
| gtgccatcat attacatcaa aatacctgcc taggaccttg cctaaggact ctgttgccgt | 11100 |
| ggtcggagtg agttcgcccg gcaaggctgc taaagccatg tgtaccctca ccgatgtgta | 11160 |
| ccttcctgaa ctccgaccat atctgcaacc aaagacggca tcaaaatgct ggaaactcaa | 11220 |
| gttagacttc agggacgtcc gactgatggt ctggaagggg gctaccgcct acttccagct | 11280 |
| tgaagggctc acatggtctg cgctgcctga ttacgccagg ttcatccagc tacccacgaa | 11340 |
| tgccgtcgtg tacatcgacc cgtgcatagg accggcagca gccaaccgta aagttgtgcg | 11400 |
| aaccacggat tggagagcgg acctggcggt aacaccatat gactacggtg ctcaaaccat | 11460 |
| tctgacgaca gcctggttcg aggacctcgg gccacagtgg aaaattttag gactacagcc | 11520 |
| cttcaggcga gcattaggct ttgacaacac tgaggactgg gcaattcttg cacgccgtat | 11580 |
| ggatgacggt aaggattaca ttgactacaa ctggagttgt gttcgaaacc gcccatgcgc | 11640 |
| tatccatgga cgtgcgcgtg accacaccta tcattttgct catggtacgg aattaggggt | 11700 |
| ggagctgggt aaacctcggt taccgcctga ataagacccc cttgaaccca aagcaatgcg | 11760 |
| atgggttcat tatggagcaa aatcagtcaa ttgttcgtgg acgcttttac cgaattcctt | 11820 |
| gttagcgtgg ttgatattgt catctttctt gccatattgt tcgggttcac agtcgctgga | 11880 |
| tggctcctgg tcttttttct cagagtggtt tgctccgcga ttctccgttc gcgctctgcc | 11940 |
| attcactctc ccgagttatc gaaggtccta tgagggcctg ctgcccaatt gcaaaccaga | 12000 |
| tgtcccacaa ttcgcaatca agcacccact aggtttgctc tggcacatgc gagtctctca | 12060 |
| gttaatcgac gagatggtct ctcgtcgcat ttaccagacc atggaacact caggtcaggc | 12120 |
| agcctggaag caggtagtca gtgaggccac ccttacaaag ttgtcaaggc tcgatgtagt | 12180 |
| tgcccatttc caacatttgg ctgcagtgga ggcggattcc tgcaacttcc tcagctcacg | 12240 |
| acttgtgatg ctgaaaaatt tagctgtcgg taacgtgagt gtattttaca acgccacgtt | 12300 |
| ggaccgcgtt gaactcgtct tccccacgcc aggcacgagg cccaaattga ccgactttag | 12360 |
| gcaatggctt atcagcgtgc atgcttccat tttctcctct gtggcttcat cggttacctt | 12420 |
| gttcatggtg ctttggcttc gggttccaac tctacgctat gtttttggtt tccattggcc | 12480 |
| cacggcaaca cgtcattcga gctgaccatc aactacacca tatgtatgcc ctgtcccacc | 12540 |
| agccaagcgg ctcaacaaag gctcgaaccc ggtcgtaata tgtggtgcaa gattggatac | 12600 |
| tctacgtgcg aggagcatga tcacgatgaa ctgtcgatgg ctataccacc tgggtatgac | 12660 |
| aatctcaaac ttgaaggcta ttatgcatgg ttggctttct tgtccttttc ttacgcggct | 12720 |
| caattccatc cggagttatt cggaataggg aatgtgtcgc gcgttttcgt ggacaagcag | 12780 |
| catcagttca tttgcgccga gcatgatgga cccaattcaa ccgtaaccac tggacataac | 12840 |
| atttccgcat tgtatgcggt gtactaccat caccaagttg acgggggcaa ctggtttcat | 12900 |
| ttagaatggc tgcggccgtt cttctcctcc tggctggtgc tcaatatctc atggtttctg | 12960 |
| aggcgttcgc ctgcaagccc tgtttctcga cgcatctatc agatattaag accaacacga | 13020 |
| ccgcggctgc cggtttcatg gtccttcaga acatcgactg cctctcagca gcgcagagga | 13080 |
| gtgctcccgt cataaagtca tctcaatgcc gtgaagccat tggtacccca cagtacatta | 13140 |
| cgataacagc aaatgtgacc gatgaagcgt atttgtacaa tgcagactta ctgatgcttt | 13200 |
| ctgcatgcct tttctatgcc tcagaactga gcgagaaagg ctttaaagtt atctttggga | 13260 |
| atatctccgg tgtcgtctct gcatgcgtca atttcacgga ttatgtgact catgtgactc | 13320 |
| aacacacaca acagcatcat ttggtgatcg atcacgttcg gttgctgcat ttcctgacac | 13380 |
| cgtcagtgat gaggtgggct acaaccatcg cttgcttact tgccatcctt ctggcgatat | 13440 |
| gagatgttct cacaaattgg ggtgtttttc gactctgtat tcctgctcct ggtggctttc | 13500 |
| tttgctgtgt accggcttgt cctggtcctt tgtcgatggc aacggcgaca gctcgacata | 13560 |
| ccaatatata tataatttga cgatatgcga gctgaatgga accaaatggt tgtccaacca | 13620 |
| ttttgattgg gcagtcgaga ctttcgtgct ttacccggtc accactcaca tcatctcatt | 13680 |
| gggtttcctc acaacaagcc atttttttga cgcgctcggt cttagcgctg tatccattgt | 13740 |
| aggatttgct gatgagcggt acgtacttag tggtgtgtac ggtgcttgtg cttttgccgc | 13800 |
| gctcgtgtgt tttgtcatcc gtgctgctaa gaactgtatg gcttgtcgct atgcccgtac | 13860 |
| ccggttcacc aatttcattg tggacgaccg gggggaaatc caccgctgga agtcaccgat | 13920 |
| agtggtggaa aagttgggca aagctgaagt cggtgatgct cttgtcacca tcaagcatgt | 13980 |
| cgtcattgaa ggggttaaag ctcaaccctt gacgagaact tcggccgagc aatggcaaac | 14040 |
| ctagatgact tctgcaatga tcccactgcc gcgcaaaagc ttgtgctagc cttcagtatt | 14100 |
| acatacacac ctataatgat atatgccctc aaagtgtcac gcggccggct cctggggctg | 14160 |
| ttgcacatcc tgatatttct gaactgttct ttcacgtttg ggtacatgac gtatgtgcat | 14220 |
| tttcaatcca ccaatcgtgt cgcgcttact atgggggctg ttgttgccct tttgtggggc | 14280 |
| atctatagtt ttacagaatc atggaggttt gtcacctcca ggtgcagact gtgttgccta | 14340 |
| ggccggcgat acattctggc ccctgcccat cacgtggaaa gtgccgcagg tctccattca | 14400 |
| atcccagcgt ctggcaaccg agcatacgct gtgagaaagc ccggactaac atcagtgaac | 14460 |
| ggcactctag taccaggact tcggagcctc gtgctgggcg gcaaacgagc tgttaaacgg | 14520 |
| ggagtggtta acctcgtcaa gtatggccgg taagaaccag agccagaaga aaaagcaaaa | 14580 |
| tacagctcct atggggaatg gccagtcagt caatcaactg tgccagctgc tgggtgtaat | 14640 |
| gatgaaatcc cagcgccaac ggcctagggg aggacaggca aaaaagaaaa agcctgagaa | 14700 |
| gccacatttc cccctggctg ctgaggatga cattcggcac cacctcaccc aaactgaacg | 14760 |
| ttctctctgc ttgcaatcga tccaaacggc tttcaaccaa ggcgcaggaa ttgcgtcgct | 14820 |
| ttcacccagc gggaaggtca gttttcaggt tgagtttatg ttgccggttg ctcatacagt | 14880 |
| gcgcttgatt cgcgtgactt ctacacccgt cagtcagggt gctagttaat ttgacagtca | 14940 |
| ggtgaatggc cgcgattgac gtgtggcctc taagtcacct attcaattag ggcgatcaca | 15000 |
| tgggggttac acttaattag gcgagaacca tgtgaccgaa att | 15043 |

A person skilled in the art would recognize the poly-adenosine tails, if any, of each of the genomic consensus sequences could vary in length from the above reported sequences.

What is claimed is:

1. A modified, live Porcine Reproductive and Respiratory Syndrome (PRRS) virus strain, wherein the consensus complementary DNA sequence of said PRRS strain is at least 95% identical to a sequence selected from the group consisting of SEQ ID NO: 1, SEQ ID NO: 2, SEQ ID NO: 3, and SEQ ID NO: 4.

2. The modified, live PRRS virus strain of claim 1, wherein the consensus complementary DNA sequence of said PRRS strain is at least 98% identical to a sequence selected from the group consisting of SEQ ID NO: 1, SEQ ID NO: 2, SEQ ID NO: 3, and SEQ ID NO: 4.

3. The modified, live PRRS virus strain of claim 1, wherein said PRRS strain is a DE 14-3073 strain designated PTA-125490; an ES 13-49 strain designated PTA-125489; an IT 14-32 strain designated PTA-125488, or a PL 14-02 strain designated PTA-125487.

4. The modified, live PRRS virus strain of claim 1, wherein said PRRS virus strain is passaged at least 85 times in tissue culture cells.

5. An immunogenic composition comprising a modified, live PRRS virus strain having a consensus complementary DNA sequence at least 90% identical to a sequence selected from the group consisting of SEQ ID NO: 1, SEQ ID NO: 2, SEQ ID NO: 3, and SEQ ID NO: 4, and a pharmaceutically-acceptable excipient, stabilizer, solubilizer, or diluent.

6. A method of treating or preventing a symptom of Porcine Reproductive and Respiratory Syndrome (PRRS) in a porcine animal, comprising administering to said porcine animal an immunogenic composition comprising a modified, live PRRS virus strain having a consensus complementary DNA sequence at least 98% identical to a sequence selected from the group consisting of SEQ ID NO: 1, SEQ ID NO: 2, SEQ ID NO: 3, and SEQ ID NO: 4.

7. The method of treating of claim 6, wherein the symptom of PRRS is caused by a PRRS virus infection in a porcine animal.


SEQUENCE LISTING (1 sequence)

SEQ ID NO: 1 - DNA, 143 bp, synthetic construct
  misc_feature 1..143: note = cDNA from Porcine Reproductive and Respiratory Syndrome virus
  source 1..143: mol_type = other DNA
atgatgtgtagggtattttccctacgtgcgcaacactttttgtgtttgtgtgcctcggaggcgtgggtatagccccgccccacctcttggccctgttctagcccaacaggtatccttctctctcggggcgagtgcgccgcctg